(19) United States
(12) Patent Application Publication
(10) Pub. No.: US 2024/0190955 A1
(43) Pub. Date: Jun. 13, 2024

(54) COMPOSITION COMPRISING AN IGE ANTIBODY

(71) Applicant: KING'S COLLEGE LONDON, London (GB)

(72) Inventors: James Spicer, London (GB); Sophia Karagiannis, London (GB)

(21) Appl. No.: 18/287,623

(22) PCT Filed: Apr. 22, 2022
(86) PCT No.: PCT/EP2022/060693

§ 371 Date: Oct. 19, 2023

(30) Foreign Application Priority Data
Apr. 23, 2021 (WO) PCT/EP2021/060749
Jul. 1, 2021 (GB) 2109550.0

(51) Int. Cl. C07K 16/28; A61K 9/00; A61K 39/00; A61P 35/00

(52) U.S. Cl. CPC C07K 16/28; A61K 9/0019; A61P 35/00; A61K 2039/505; A61K 2039/545; C07K 2317/24; C07K 2317/732

(57) ABSTRACT

In one aspect, the present invention relates to an anti-folate receptor alpha (FRα) immunoglobulin E (IgE) antibody for use in treating a low FRα-expressing tumor in a subject.

### FIELD OF THE INVENTION

The present invention relates to the field of therapeutic antibodies and uses thereof and in particular to immunoglobulin E (IgE) antibodies for use in treating cancer. The present invention also relates to methods of treating diseases such as cancer using such IgE antibodies.

### BACKGROUND

Therapeutic antibodies now complement conventional treatments for a number of malignant diseases, but almost all agents currently developed rely on only one of the nine human antibody classes, namely IgG₁, the most abundant antibody class in the blood (Weiner L M, Surana R, Wang S (2010) Monoclonal antibodies: versatile platforms for cancer immunotherapy. Nat Rev Immunol 10: 317-327). The human immune system naturally deploys nine antibody classes and subclasses (IgM, IgD, IgG1-4, IgA1, IgA2 and IgE) to perform immune surveillance and to mediate destruction of pathogens in different anatomical compartments. Yet only IgG (most often IgG1) has been applied in immunotherapy of cancers.

One reason may be that IgG antibodies (particularly IgG1), constitute the largest fraction of circulating antibodies in human blood. The choice of antibody class is also based on pioneering work in the late 1980s, comparing a panel of chimaeric antibodies of the same specificity, each with Fc regions belonging to one of the nine antibody classes and subclasses (Bruggemann M, Williams G T, Bindon C I, Clark M R, Walker M R, Jefferis R, Waldmann H, Neuberger M S (1987) Comparison of the effector functions of human immunoglobulins using a matched set of chimeric antibodies. J Exp Med 166: 1351-1361). Antibodies were evaluated for their ability to bind complement and their potency to mediate haemolysis and cytotoxicity of antigen-expressing target cells in the presence of complement. IgG1 in combination with human peripheral blood mononuclear cells (PBMC) was the most effective IgG subclass in complement-dependent cell killing in vitro, while the IgA and IgE antibodies were completely inert.

Subsequent clinical trials with antibodies recognising the B cell marker CD20 supported the inference that IgG1 would be the subclass best suited for immunotherapy of patients with B cell malignancies such as non-Hodgkin's lymphoma (Alduaij W, Illidge T M (2011) The future of anti-CD20 monoclonal antibodies: are we making progress? Blood 117: 2993-3001). Since those studies, comparisons of anti-tumour effects by different antibody classes have been confined to IgG and IgM in both murine models and patients with lymphoid malignancies, while IgA has been shown to mediate ADCC in vitro and in vivo in mouse models of lymphoma (Dechant M, Valerius T (2001) IgA antibodies for cancer therapy. Crit Rev Oncol Hematol 39: 69-77).

Folate receptor α (FRα) is a cancer-associated antigen that is over-expressed in several solid cancer types (including ovarian and endometrial cancer and mesothelioma). Expression of FRα has been described in in normal kidney, placenta, lung, Fallopian tube, pancreas and testis, but not in other normal tissues, including the heart, liver, spleen, gastrointestinal tract, ovary, uterus, muscle, lymphoid and glandular tissues (Weitman, Lark et al., Cancer Res 52(12): 3396-3401; Kelemen 2006, Int J Cancer 119(2): 243-250). In normal tissues, the level of FRα expression is generally low or restricted to luminal surfaces and is therefore unlikely to be accessible to circulating antibodies (Parker, Turk et al. 2005, Anal Biochem 338(2): 284-293; O'Shannessy, Yu et al. 2012, Oncotarget 3(4): 414-425). FRα expression is found in up to 40% of primary ovarian and endometrial tumours, and nearly 30% of lung cancers. FRα in tumours (particularly ovarian and endometrial cancers) is known to be accessible to antibodies and may be expressed at much greater levels than in normal tissues (Antony 1996, Annu. Rev. Nutr. 16: 501-521). Due to the different level and location of FRα expression in normal tissues and tumours, FRα is therefore considered to be an effectively tumour-specific antigen (Mantovani, Miotti et al. 1994, Eur J Cancer 30A(3): 363-369; Toffoli, Cernigoi et al. 1997, Int J Cancer 74(2): 193-198).

Initial clinical trials targeting FRα using IgG antibodies suggested good tolerability. For instance, farletuzumab (MORAb-003), a humanised anti-FRα IgG antibody was well tolerated at doses ranging from 12 mg/m² to 40 mg/m² in a Phase I study in platinum-resistant epithelial ovarian carcinoma, (see e.g. Konner et al., Clin Cancer Res. 2010 Nov. 1; 16(21):5288-95). A number of anti-FRα IgG antibodies are in development, including antibody-drug conjugates (ADCs). For instance, mirvetuximab soravtansine is an ADC consisting of an FRα-binding antibody, a cleavable linker, and cytotoxic payload that has been used in trials for ovarian cancer.

However Phase III clinical trials of anti-FRα IgG therapy have suggested that high FRα expression may be required for efficacy of these antibodies. For instance, a Phase III trial of farletuzumab in platinum-resistant epithelial ovarian carcinoma failed to achieve its primary objective, i.e. improved progression-free survival (Vergote et al., Int J Gynecol Cancer. 2013; 23(8 Suppl 1):11; Walters et al., Gynecol Oncol. 2013; 131(2):493-498). It was suggested that this lack of efficacy could be due to a failure to include a minimum level of FRα expression as an eligibility criterion in this trial (Sato and Itamochi, OncoTargets and Therapy 2016:9 1181-1188).

Moreover a Phase III trial (FORWARD I) of ImmunoGen's mirvetuximab soravtansine for FRα-positive platinum-resistant ovarian cancer failed to meet its primary endpoint (see Immunogen press release dated Mar. 1, 2019, ImmunoGen Announces Top-Line Results from Phase 3 FORWARD I Study of Mirvetuximab Soravtansine in Ovarian Cancer, available at https://investor.immunogen.com/news-releases/news-release-details/immunogen-announces-top-line-results-phase-3-forward-i-study).

Eligibility criteria for the FORWARD I trial included patients with platinum-resistant ovarian cancer that expressed medium or high levels of FRα who have been treated with up to three prior regimens. It was suggested that high levels of FRα expression might be required for efficacy (see Immunogen press release dated Sep. 29, 2019, ImmunoGen Presents Full Data from Phase 3 FORWARD I Study of Mirvetuximab Soravtansine in Ovarian Cancer at ESMO, available at https://investor.immunogen.com/news-releases/news-release-details/immunogen-presents-full-data-phase-3-forward-i-study). Therefore a further Phase III trial (SORAYA) focused on high expressor patients only and had a more specific diagnostic test (see ClinicalTrials.gov Identifier: NCT04296890, A Study of Mirvetuximab Soravtansine in Platinum-Resistant, Advanced High-Grade Epithelial Ovarian, Primary Peritoneal, or Fallopian Tube Cancers With High Folate Receptor-Alpha Expression (SORAYA)). Other studies of mirvetuximab soravtansine in combination with further chemotherapeutic agents have also suggested high FRα expression is required (see e.g. Cristea et al., A phase I study of mirvetuximab soravtansine (MIRV) and gemcitabine (G) in patients (Pts) with selected FRα-positive solid tumors: Results in the ovarian cancer (EC) cohort; Journal of Clinical Oncology 2021 39:15_suppl, 5542).

Similarly, the anti-FRα antibody MOv18-IgG1 has been shown to induce tumor cell killing in high FRα-expressing, but not of low FRα-expressing, cancer cells by a combination of by Antibody-Dependent Cell-mediated Cytotoxicity (ADCC) and Phagocytosis (ADCP) functions (Cheung et al., Clin Cancer Res. 2018 Oct. 15; 24(20): 5098-5111). The authors of Cheung et al. suggest that the lack of cell-mediated killing by MOv18-IgG1 against low FRα-expressing cells may be important in avoiding on-target/off-tumor toxic effects. There is accordingly a need for improved treatments for tumors, e.g. in ovarian cancer, that do not express high levels of FRα.

Antibodies of the IgE class play a central role in allergic reactions and have many properties that may be advantageous for cancer therapy. IgE-based active and passive immunotherapeutic approaches have been shown to be effective in both in vitro and in vivo models of cancer, suggesting the potential use of these approaches in humans (Leoh et al., Curr Top Microbiol Immunol. 2015; 388: 109-149). Thus IgE therapeutic antibodies may offer enhanced immune surveillance and superior effector cell potency against cancer cells.

A mouse/human chimeric IgE antibody (MOv18 IgE), which is specific for FRα, has been demonstrated to have superior antitumor efficacy compared with an otherwise identical IgG in a syngeneic immunocompetent animal (Gould et al., Eur J Immunol 1999; 29:3527-37; Josephs et al., Cancer Res. 2017 Mar. 1; 77(5):1127-1141; Karagiannis et al., Cancer Res. 2017 Jun. 1; 77(11):2779-2783). TNFα/MCP-1 signaling was identified as an IgE-mediated mechanism of monocyte and macrophage activation and recruitment to tumors. These findings draw parallels with powerful macrophage-activating functions employed by IgE against parasites, rather than allergic IgE mechanisms. The potential clinical application of IgE-derived drugs in clinical oncology is clear if the antitumor activity of MOv18 IgE in these preclinical experiments can be replicated in patients.

However the absence of clinical trial data relating to the therapeutic use of IgE antibodies in humans means that appropriate methods of treatment and uses involving IgE antibodies are still lacking. In particular, it is not known in which sub-groups of cancer patients therapeutic IgE antibodies may be effective. Since anti-FRα IgG antibodies are indicated primarily for high FRα expressors, there is a particular need for improved treatments for other sub-groups of cancer patients, including in ovarian cancer. However it is not known how methods and uses developed for administration of other therapeutic antibody isotypes (e.g. IgG) could be adapted for IgE antibodies, nor whether IgG and IgE antibodies could be used to treat the same or different sub-groups of patients.

### SUMMARY OF THE INVENTION

Accordingly, in one aspect the present invention provides an anti-folate receptor alpha (FRα) immunoglobulin E (IgE) antibody for use in treating a low FRα-expressing tumor in a subject. In one embodiment, by “low FRα-expressing tumor” it is meant that less than 50% of tumor cells in the subject express FRα. Preferably less than 40%, 30%, 20% or 10% of tumor cells in the subject express FRα.

In another embodiment, less than 50% of tumor cells in the subject show detectable FRα expression, e.g. detectable membrane (i.e. cytoplasmic membrane) FRα expression. Preferably less than 50%, 40%, 30%, 25%, 20% or 10% of tumor cells in the subject show detectable membrane FRα expression. Expression (e.g. membrane expression) of FRα is typically detected using immunohistochemistry, i.e. detectable expression refers to immunohistochemical detection of FRα.

In another embodiment, less than 50%, 40%, 30%, 25%, 20% or 10% of tumor cells in the subject show moderate- or high- (2+) intensity staining for (e.g. membrane) FRα, typically when using immunohistochemical detection of FRα.

In another embodiment, tumor cells in the subject are classified according to membrane FRα staining intensity. FRα staining intensity may be classified on a scale from 0 (no detectable FRα) to 3 (high FRα staining intensity), e.g. wherein 1 indicates low FRα staining intensity and and 2 indicates moderate FRα staining intensity. In some embodiments, tumor cells in the subject are further classified according to a percentage of tumor cells positive for FRα, (e.g. from 0 to 100%).

Preferably an overall tumor FRα expression score for the subject is determined as the product of the membrane FRα staining intensity score and the percentage of tumor cells positive for FRα. For instance, in some embodiments the overall tumor FRα expression score for the subject may be less than 100, preferably less than 90, 80, 70, 60, 50 or 40, more preferably less than 30 or less than 20.

In alternative embodiments, tumor FRα expression in the subject may be compared to that in other cancer subjects, e.g. other subjects suffering from the same type of cancer. Thus the relative level of tumor FRα expression in the subject may be ascertained. In preferred embodiments, tumor (e.g. membrane) FRα expression in the subject is lower than in at least 50% of cancer subjects. Preferably (e.g. membrane) FRα expression in tumor cells of the subject is lower than in at least 60%, at least 70% or at least 80% of cancer subjects, e.g. suffering from the same form of cancer (preferably ovarian cancer). More preferably tumor (e.g. membrane) FRα expression in the subject is lower than in at least 50%, 60%, 70%, 80% or at least 90% of FRα-expressing tumors (preferably FRα-expressing ovarian tumors).

In preferred embodiments the tumor expresses FRα, i.e. the tumor shows at least some FRα expression. Preferably tumor cells in the subject show detectable membrane FRα expression, e.g. by immunohistochemistry. More preferably at least 1% or at least 5% of tumor cells in the subject show detectable (e.g. membrane) FRα expression, e.g. using immunohistochemical detection of FRα. In other embodiments at least 10%, 15% or 20% of tumor cells in the subject show detectable membrane FRα expression.

In one embodiment, the antibody is a MOv18 IgE antibody.

In one embodiment, IgE antibody is used to treat and/or delay progression of cancer in the subject. For instance the antibody may be used to delay progression of the low FRα-expressing tumor in a subject.

In one embodiment, the tumor or cancer is an ovarian tumor or ovarian cancer.

In one embodiment the antibody lacks a cytotoxic moiety. Thus the antibody may comprise, consist of or consist essentially of (only) one or more (e.g. four) polypeptide chains, e.g. immunoglobulin (preferably IgE) heavy and/or light chains. In particular, it is preferred that the antibody is not an antibody-drug conjugate (ADC). Thus the antibody may lack a further drug or group having a cytotoxic effect, e.g. a chemotherapeutic or drug that is capable of (directly) killing cancer cells. The antibody may further lack a linker or other group for conjugating a cytotoxic moiety to a polypeptide.

In one embodiment, a weekly dose of the IgE antibody administered to the subject is less than 50 mg, 25 mg, 10 mg, 3 mg or 1 mg. In another embodiment, the weekly dose of the IgE antibody is 10 μg to 50 mg, 70 μg to 30 mg, 70 μg to 3 mg, 500 μg to 1 mg or about 700 μg.

Preferably the IgE antibody is administered to the subject once a week or once every two weeks. In one embodiment, the IgE antibody is administered to the subject for up to 12 weeks. In another embodiment, the IgE antibody is administered to the subject (i) once a week for 6 weeks; followed by (ii) once every two weeks for 6 weeks.

In one embodiment, the IgE antibody is administered to the subject in a dose per administration of less than 1 mg/kg, less than 0.1 mg/kg or less than 0.03 mg/kg. In another embodiment, the IgE antibody is administered to the subject in a dose of less than 1 mg/kg/week, less than 0.1 mg/kg/week, or less than 0.03 mg/kg/week.

In a further aspect, the present invention provides a method for treating and/or delaying progression of cancer in a subject having a low FRα-expressing tumor, the method comprising a step of administering an anti-folate receptor alpha (FRα) immunoglobulin E (IgE) antibody as defined in any preceding claim to the subject in a therapeutically-effective amount.

In a further aspect, the present invention provides a pharmaceutical composition for use in treating a low FRα-expressing tumor in a subject, comprising an anti-folate receptor alpha (FRα) immunoglobulin E (IgE) antibody as defined above and one or more pharmaceutically acceptable excipients, carriers or diluents.

Preferably the composition comprises less than 50 mg of the IgE antibody. More preferably the composition comprises less than 30 mg, less than 25 mg, less than 10 mg, less than 5 mg, less than 3 mg or less than 1 mg of the IgE antibody. In other embodiments, the composition comprises 10 μg to 50 mg, 70 μg to 30 mg, 70 μg to 3 mg, 500 μg to 1 mg or about 700 μg of the IgE antibody.

In one embodiment, the composition is in the form of a liquid. Preferably the composition is an aqueous solution having a concentration of 0.1 mg/ml to 10 mg/ml, 0.5 mg/ml to 2 mg/ml or about 1 mg/ml of the IgE antibody. Preferably the pharmaceutically acceptable excipient is selected from sodium citrate, L-arginine, sucrose, polysorbate 20 and/or sodium chloride.

In one embodiment, the composition is suitable for intravenous injection or subcutaneous injection. Preferably the composition is suitable for intravenous or subcutaneous injection up to a maximum total dose of 50 mg/week, 25 mg/week, 10 mg/week, 3 mg/week or 1 mg/week.

### BRIEF DESCRIPTION OF THE DRAWINGS

FIG. 1 shows the amino acid sequence of MOv18 IgE Light (L) Chain (SEQ ID NO:1); the mouse VL in shown in bold and the human CL is shown in standard text.

FIG. 2 shows the amino acid sequence of MOv18 IgE Heavy (H) Chain (SEQ ID NO:2); the mouse VH is shown in bold and the human CH is shown in standard text.

FIG. 3 shows the amino acid sequence of MOv18 IgE light chain variable domain (VL) (SEQ ID NO:3).

FIG. 4 shows the amino acid sequence of MOv18 IgE heavy chain variable domain (VH) (SEQ ID NO:4).

FIG. 5 shows the pharmacokinetics (serum concentration) of MOv18 IgE following intravenous administration of the antibody.

FIG. 6 shows a CT scan image and the results of tumor measurements taken from the CT scan image indicating a reduction in tumor size in an ovarian cancer subject treated with the 700 μg dose level of MOv18 IgE antibody. The tumor (shown in area within oval on each image) is depicted at baseline (left panel) and after 6 weeks of treatment (right panel). The target and non-target lesion dimensions and status in the subject were determined before and after cycles of treatment with the antibody and after the maintenance period.

FIG. 7 shows a significant decrease in serum concentration of the ovarian cancer antigen CA125 during treatment of a patient with 6 weekly doses of 700 μg MOv18 IgE antibody, followed by 3 further 700 μg doses of the antibody at 2 week intervals.

FIG. 8 shows a plot of the change in RECIST (Response Evaluation Criteria in Solid Tumours) scores in individual ovarian cancer subjects treated with a MOv18 IgE antibody. Each line represents the percentage change in RECIST scores in individual patients from the start of treatment, (i.e. after 6 weeks of treatment and after 12 weeks of treatment). A RECIST score that increases or decreases by less than 20% is indicative of stable disease.

FIG. 9 shows a waterfall plot of the change in RECIST (Response Evaluation Criteria in Solid Tumours) scores in individual ovarian cancer subjects after 6 weeks of treatment with a MOv18 IgE antibody. Each vertical bar represents the change in RECIST score in an individual subject at 6 weeks. 20 subjects in total were treated. Where no vertical bar is shown, this indicates no change in the RECIST score in the subject after 6 weeks (this occurred in four subjects, indicated by the gap between vertical bars along the x-axis).

FIG. 10 shows a waterfall plot of the change in RECIST (Response Evaluation Criteria in Solid Tumours) scores in individual ovarian cancer subjects after 6 or 12 weeks of treatment with a MOv18 IgE antibody. The same subjects as shown in FIG. 9 are represented in the same order. Only some (5) subjects continued with treatment beyond 6 weeks. Each vertical bar marked with an asterisk (*) represents the change in RECIST score in an individual subject treated to 12 weeks. The remaining vertical bars without asterisks represent the change in RECIST scores in individual subjects treated to 6 weeks, as shown in FIG. 9. Where no vertical bar is shown, this indicates a change in RECIST score of 0% in the subject (this occurred in two subjects, indicated by the gap in vertical bars along the x-axis).

FIG. 11 shows a waterfall plot of the change in RECIST (Response Evaluation Criteria in Solid Tumours) scores in individual ovarian cancer subjects after 6 weeks of treatment with a MOv18 IgE antibody, compared to the FRα expression score in each subject. Each vertical bar represents the change in RECIST score in an individual subject at 6 weeks. 20 subjects in total were treated. Where no vertical bar is shown, this indicates no change in the RECIST score in the subject after 6 weeks (this occurred in four subjects, indicated by the gap between vertical bars along the x-axis). PD indicates progressive disease and SD indicates stable disease. The overall FRα expression score for each subject is computed as the product of a membrane staining intensity score (Membrane score) and the percentage of tumor cells positive for FRα (% membrane+).

### DETAILED DESCRIPTION OF THE INVENTION

It has surprisingly been found that anti-FRα IgE antibodies can provide an effective treatment for low FRα-expressing tumors. In particular, an anti-FRα IgE (MOv18 IgE) was found to be effective in treating or delaying progression of ovarian cancer in subjects having a very low FRα membrane expression score.

This finding is particularly surprising because the equivalent IgG antibody (i.e. MOv18 IgG1) was known to kill high FRα-expressing but not low FRα-expressing tumors, and this selectivity was considered to be important in avoiding off-tumor toxic effects (Cheung et al., Clin Cancer Res. 2018 Oct. 15; 24(20): 5098-5111). Moreover, anti-FRα treatment approaches using IgG antibodies have focused on treating high FRα expressors and/or combination of the antibody with a cytotoxic moiety, e.g. in an ADC and/or a separate combination therapy with agents such as gemcitabine (see Martin et al. Gynecol Oncol. 2017 November; 147(2): 402-407; Sato and Itamochi, OncoTargets and Therapy 2016:9 1181-1188 and Cristea et al., Journal of Clinical Oncology 2021 39:15_suppl, 5542).

In contrast, anti-FRα IgE antibodies are capable of treating low FRα-expressing tumors as a monotherapy, i.e. without incorporation into an ADC or combination with a further chemotherapeutic drug. Therefore the present invention provides a significant contribution to the art in addressing an unmet medical need, specifically in a sub-group of cancer patients who are low FRα expressors.

Moreover, it has been found that the methods, compositions and dosage forms and regimens that are typically used for IgG antibodies are not necessarily suitable for IgE antibodies. In particular, it has been demonstrated herein that the minimum dose of IgE antibodies required for efficacy (e.g. for an anti-tumour effect in a low FRα-expressing subject) can be very much lower than a typical effective dose for IgG antibodies. For instance, as shown in the Example below, an anti-folate receptor α (FRα) IgE antibody was found to have anti-tumour effects at a unit dose as low as 700 μg (approx. 0.01 mg/kg), which is several orders of magnitude lower than atypical IgG therapeutic antibody dose (e.g. around 150-2000 mg per dose or 2-20 mg/kg).

This result shows that due to the differences in the pharmacology and pharmacokinetics of IgG and IgE, methods, uses and compositions (such as dosage regimens, unit dosage forms and subgroups of cancer patients to be treated) developed for IgG antibodies are not necessarily transferrable to IgEs. The present inventors therefore developed new uses and dosage regimens that are particularly applicable to therapeutic IgE administration, e.g. in the treatment of cancer in low FRα-expressing patients.

### Therapeutic Antibody

Antibodies are polypeptide ligands comprising at least a light chain or heavy chain immunoglobulin variable region which specifically recognizes and specifically binds an epitope of an antigen, such as FRα, or a fragment thereof. Antibodies are typically composed of a heavy and a light chain, each of which has a variable region, termed the variable heavy (VH) region and the variable light (VL) region. Together, the VH region and the VL region are responsible for binding the antigen recognized by the antibody.

Antibodies include intact immunoglobulins and the variants and portions of antibodies well known in the art, provided that such fragments retain at least one function of IgE, e.g. are capable of binding an Fcε receptor. Antibodies also include genetically engineered forms such as chimaeric, humanized (for example, humanized antibodies with murine sequences contained in the variable regions) or human antibodies, heteroconjugate antibodies (such as, bispecific antibodies), e.g. as described in Kuby, J., Immunology, 3rd Ed., W.H. Freeman & Co., New York, 1997.

Typically, a naturally occurring immunoglobulin has heavy (H) chains and light (L) chains interconnected by disulfide bonds. There are two types of light chain, lambda (k) and kappa (κ). There are nine main isotypes or classes which determine the functional activity of an antibody molecule: IgA1-2, IgD, IgE, IgG1-4 and IgM, corresponding to the heavy chain types α, δ, ε, γ, and μ. Thus, the type of heavy chain present defines the class of antibody. Distinct heavy chains differ in size and composition; α and γ contain approximately 450 amino acids, while μ and ε have approximately 550 amino acids. The differences in the constant regions of each heavy chain type account for the different effector functions of each antibody isotype, by virtue of their selective binding to particular types of receptor (e.g. Fc receptors). Accordingly, in embodiments of the present invention the antibody preferably comprises an epsilon (c) heavy chain, i.e. the antibody is of the isotype IgE which binds to Fcε receptors.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs.” The extent of the framework region and CDRs has been defined (see, Kabat et al., Sequences of Proteins of Immunological Interest, U.S. Department of Health and Human Services, 1991). The Kabat database is now maintained online. The sequences of the framework regions of different light or heavy chains are relatively conserved within a species, such as humans. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found.

Antibodies may have a specific VH region and the VL region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). References to “VH” refer to the variable region of an immunoglobulin heavy chain. References to “VL” refer to the variable region of an immunoglobulin light chain.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “chimaeric antibody” comprises sequences derived from two different antibodies, which are typically derived from different species. For example, chimaeric antibodies may include human and murine antibody domains, e.g. human constant regions and murine variable regions (e.g. from a murine antibody that specifically binds to a target antigen).

Chimaeric antibodies are typically constructed by fusing variable and constant regions, e.g. by genetic engineering, from light and heavy chain immunoglobulin genes belonging to different species. For example, the variable segments of the genes from a mouse monoclonal antibody can be joined to human constant segments, such as kappa and epsilon. In one example, a therapeutic chimaeric antibody is thus a hybrid protein composed of the variable or antigen-binding domain from a mouse antibody and the constant or effector domain from a human antibody, e.g. an Fc (effector) domain from a human IgE antibody, although other mammalian species can be used, or the variable region can be produced by molecular techniques. Methods of making chimaeric antibodies are well known in the art, e.g., see U.S. Pat. No. 5,807,715.

A “humanized” antibody is an antibody including human framework regions and one or more CDRs from a non-human (for example a mouse, rat, or synthetic) antibody. The non-human immunoglobulin providing the CDRs is termed a “donor”, and the human immunoglobulin providing the framework is teamed an “acceptor”. In one embodiment, all the CDRs are from the donor immunoglobulin in a humanized immunoglobulin. The constant regions are typically substantially identical to human immunoglobulin constant regions, i.e., at least about 85-90%, such as about 95% or more identical. Hence, all parts of a humanized immunoglobulin, except the CDRs, are substantially identical to corresponding parts of natural human immunoglobulin sequences.

A humanized antibody typically comprises a humanized immunoglobulin light chain and a humanized immunoglobulin heavy chain. A humanized antibody typically binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions.

Humanized immunoglobulins can be constructed by means of genetic engineering (see for example, U.S. Pat. No. 5,585,089). Typically humanized monoclonal antibodies are produced by transferring donor antibody complementarity determining regions from heavy and light variable chains of a mouse immunoglobulin into a human variable domain, and then substituting human residues in the framework regions of the donor counterparts. The use of antibody components derived from humanized monoclonal antibodies obviates potential problems associated with the immunogenicity of the constant regions of the donor antibody. Techniques for producing humanized monoclonal antibodies are described, for example, by Jones et al., Nature 321:522, 1986; Riechmann et al., Nature 332:323, 1988; Verhoeyen et al., Science 239:1534, 1988; Carter et al., Proc. Nat'l Acad. Sci. U.S.A. 89:4285, 1992; Sandhu, Crit. Rev. Biotech. 12:437, 1992; and Singer et al., J. Immunol. 150:2844, 1993.

A “human” antibody (also called a “fully human” antibody) is an antibody that includes human framework regions and all of the CDRs from a human immunoglobulin. In one example, the framework and the CDRs are from the same originating human heavy and/or light chain amino acid sequence. However, frameworks from one human antibody can be engineered to include CDRs from a different human antibody.

In embodiments of the present invention, the antibodies may be monoclonal or polyclonal antibodies, including chimaeric, humanized or fully human antibodies.

### Anti-FRα Antibodies

In some embodiments, the antibody binds specifically to folate receptor a (FRα) to form an immune complex. Typically the antibody may comprise an antigen-binding region (e.g. one or more variable regions, or one to 6 CDRs) derived from an antibody which is known to bind. FRα, preferably human FRα, e.g. MOv18 IgE.

FRα (also known as folate receptor 1 or FOLR1), is over-expressed in several solid cancer types (including ovarian and endometrial cancer and mesothelioma). The antigen has been characterised as effectively tumour specific and clinical trials targeting FRα using IgG and IgE antibodies have demonstrated favourable tolerability profiles. The MOv18 IgG and IgE antibodies which bind to FRα and their properties are described, for example, in Coney, L. R., A. Tomassetti, et al. (1991). Cancer Res 51(22): 6125-6132; Gould, H. J., G. A. Mackay, et al. (1999). Eur J Immunol 29(11): 3527-3537; Karagiannis, S. N., Q. Wang, et al. (2003). Eur J Immunol 33(4): 1030-1040.

In one specific embodiment, the antibody comprises a variable region (e.g. a heavy chain variable domain (VH) and/or a light chain variable domain (VL)) or at least one, two, three, four, five or six CDRs (e.g. 3 heavy chain CDRs or 3 light chain CDRs) from MOv18 IgG or IgE, e.g. the CDRs present in SEQ ID NO:4 and/or SEQ ID NO:3, wherein the CDR sequences may be defined according to the method of Kabat, Chothia or IMGT (see e.g. Dondelinger, Front Immunol. 2018; 9: 2278 and references cited therein, which are incorporated herein by reference). For instance, CDRs may be defined according to Kabat: see Kabat E A, et al. (U.S.) NI of H. Sequences of Immunoglobulin Chains: Tabulation Analysis of Amino Acid Sequences of Precursors, V-regions, C-regions, J-Chain BP-Microglobulins, 1979; or according to Chothia: see Chothia C, et al, Canonical structures for the hypervariable regions of immunoglobulins, J Mol Biol. 1987 Aug. 20; 196(4):901-1; or according to IMGT: see Giudicelli V et al., IMGT, the international ImMunoGeneTics database, Nucleic Acids Res. 1997 Jan. 1; 25(1):206-11 or Lefranc M P, Unique database numbering system for immunogenetic analysis, Immunol Today. 1997 November; 18(11):509. The amino acid sequences of the VH and VL domains of MOv18 IgE are shown in SEQ ID NO:4 and SEQ ID NO:3, respectively. In another embodiment, the antibody is a chimaeric, humanized or fully human antibody that specifically binds the epitope bound by MOv18 IgE. Most preferably the therapeutic antibody is MOv18 IgE, e.g. the antibody comprises a light chain amino acid sequence as defined in SEQ ID NO:1 and/or a heavy chain amino acid sequence as defined in SEQ ID NO:2.

In another example, the antibody comprises a variable region (e.g. a heavy chain variable domain and/or a light chain variable domain) or at least one, two, three, four, five or six CDRs (e.g. 3 heavy chain CDRs or 3 light chain CDRs) derived from a human B cell clone that recognises an epitope found on e.g. FRα, preferably human FRα.

In one embodiment, the antibody comprises one or more human constant regions, e.g. one or more human heavy chain constant domains (e.g. ε constant domains) and/or a human light chain (e.g. κ or λ) constant domain. An amino acid sequence of a human light (κ) chain constant domain is shown in SEQ ID NO:1 (non-bold text). An amino acid sequence of a human heavy chain constant domain is shown in SEQ ID NO:2 (non-bold text). In one embodiment the antibody comprises one or more human framework regions within the VH and/or VL domains. In one embodiment, the sequence of a humanized immunoglobulin heavy chain variable region framework can be at least about 65% identical to the sequence of the donor immunoglobulin heavy chain variable region framework. Thus, the sequence of the humanized immunoglobulin heavy chain variable region framework can be at least about 75%, at least about 85%, at least about 99% or at least about 95%, identical to the sequence of the donor immunoglobulin heavy chain variable region framework. Human framework regions, and mutations that can be made in a humanized antibody framework regions, are known in the art (see, for example, U.S. Pat. No. 5,585,089).

Further antibodies against a specific antigen, e.g. FRα, may also be generated by well-established methods, and at least the variable regions or CDRs from such antibodies may be used in the antibodies of the present invention (e.g. the generated antibodies may be used to donate CDR or variable region sequences into IgE acceptor sequences). Methods for synthesizing polypeptides and immunizing a host animal are well known in the art. Typically, the host animal (e.g. a mouse) is inoculated intraperitoneally with an amount of immunogen (e.g. FRα or a polypeptide comprising an immunogenic fragment thereof), and (in the case of monoclonal antibody production) hybridomas prepared from its lymphocytes and immortalized myeloma cells using the general somatic cell hybridization technique of Kohler, B. and Milstein, C. (1975) Nature 25 6:495-497.

The sequence of human FRα is well known (see e.g. UniProt database accession no. P15328) and thus human FRα may, for example, be purified from a natural source or expressed using recombinant techniques for use in such methods. The amino acid and nucleic acid sequences of human FRα are shown below in SEQ ID NO:s 5 and 6 respectively:

Human FRa amino acid sequence:
SEQ ID NO: 5
MAQRMTTQLLLLLVWVAVVGEAQTRIAWARTELLNVCMNAKHHKE
KPGPEDKLHEQCRPWRKNACCSTNTSQEAHKDVSYLYRFNWNHCG
EMAPACKRHFIQDTCLYECSPNLGPWIQQVDQSWRKERVLNVPLC
KEDCEQWWEDCRTSYTCKSNWHKGWNWTSGFNKCAVGAACQPFHF
YFPTPTVLCNEIWTHSYKVSNYSRGSGRCIQMWFDPAQGNPNEEV
ARFYAAAMSGAGPWAAWPFLLSLALMLLWLLS
Human FRα nucleic acid sequence:
SEQ ID NO: 6
atggctcagcggatgacaacacagctgctgctccttctagtgtgg
gtggctgtagtaggggaggctcagacaaggattgcatgggccagg
actgagcttctcaatgtctgcatgaacgccaagcaccacaaggaa
aagccaggccccgaggacaagttgcatgagcagtgtcgaccctgg
aggaagaatgcctgctgttctaccaacaccagccaggaagcccat
aaggatgtttcctacctatatagattcaactggaaccactgtgga
gagatggcacctgcctgcaaacggcatttcatccaggacacctgc
ctctacgagtgctcccccaacttggggccctggatccagcaggtg
gatcagagctggcgcaaagagcgggtactgaacgtgcccctgtgc
aaagaggactgtgagcaatggtgggaagattgtcgcacctcctac
acctgcaagagcaactggcacaagggctggaactggacttcaggg
tttaacaagtgcgcagtgggagctgcctgccaacctttccatttc
tacttccccacacccactgttctgtgcaatgaaatctggactcac
tcctacaaggtcagcaactacagccgagggagtggccgctgcatc
cagatgtggttcgacccagcccagggcaaccccaatgaggaggtg
gcgaggttctatgctgcagccatgagtggggctgggccctgggca
gcctggcctttcctgcttagcctggccctaatgctgctgtggctg
ctcagc

Hybridomas that produce suitable antibodies may be grown in vitro or in vivo using known procedures. Monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. If desired, the antibody (monoclonal or polyclonal) of interest may be sequenced and the polynucleotide sequence may then be cloned into a vector for expression or propagation. The sequence encoding the antibody may be maintained in a vector in a host cell and the host cell can then be expanded and frozen for future use.

Phage display technology, for instance as described in U.S. Pat. No. 5,565,332 and other published documents, may be used to select and produce human antibodies and antibody fragments in vitro, from immunoglobulin variable (V) domain gene repertoires from unimmunized donors (e.g. from human subjects, including patients suffering from a relevant disorder). For example, existing antibody phage display libraries may be panned in parallel against a large collection of synthetic polypeptides. According to this technique, antibody V domain genes are cloned in frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus antibody sequences selected using phage display from human libraries may include human CDR or variable region sequences conferring specific binding to a specific antigen such as FRα, which may be used to provide fully human antibodies for use in the present invention.

Methods for deriving heavy and light chain sequences from human B cell and plasma cell clones are also well known in the art and typically performed using polymerase chain reaction (PCR) techniques, examples of the methods are described in: Kuppers R, Methods Mol Biol. 2004; 271:225-38; Yoshioka M et al., BMC Biotechnol. 2011 Jul. 21; 11:75; Scheeren F A et al., PLoS ONE 2011, 6(4): e17189. doi:10.1371/journal.pone.0017189; Wrammert J et al., Nature 2008 453, 667-671; Kurosawa N et al., BMC Biotechnol. 2011 Apr. 13; 11:39; Tiller et al., J Immunol Methods. 2008 Jan. 1; 329(1-2): 112-124. Thus antibody sequences selected using B cell clones may include human CDR or variable region sequences conferring specific binding to e.g. FRα, which may be used to provide fully human antibodies for use in the present invention.

### IgE Antibodies

The therapeutic antibody to be administered to the subject is an IgE antibody, i.e. an antibody of the isotype IgE. There are some fundamental structural differences between IgEs and IgGs, and these have functional effects. While IgE shares the same basic molecular architecture as antibodies of other classes, the heavy chain of IgE contains one more domain than the heavy chain of IgG. The Cε3 and Cε4 domains of IgE are homologous in sequence, and similar in structure, to the Cγ2 and Cγ3 domains of IgG, so that it is the Cε2 domains that are the most obvious distinguishing feature of IgE. The Cε2 domain has been found to be folded back against the heavy chain IgE and to make extensive contact with the Cε3 domain. This bent structure of the IgE heavy chain allows it to adopt an open or closed conformation. The unbound IgE dimer has one chain in the open and one chain in the closed conformation. Binding of FcεRI to IgE is biphasic and is thought to involve initial binding to the open CF chain followed by extensive structural rearrangement to allow binding to the closed CF chain. The binding between the IgE dimer and the FcεRI occurs with 1:1 stoichiometry despite the presence of two identical CF-chains. This rearrangement results in a very tight interaction between IgE and FcεRI, and a much greater affinity of IgE for its Fc receptor than found with IgG and FcγRs (McDonnell, J. M., R. Calvert, et al. (2001) Nat Struct Biol 8(5): 437-441).

The antibodies used in the present invention are typically capable of binding to Fcε receptors, e.g. to the FcεRI and/or the FcεRII receptors. Preferably the antibody is at least capable of binding to FcεRI (i.e. the high affinity Fcε receptor) or is at least capable of binding to FcεRII (CD23, the low affinity Fcε receptor). Typically the antibodies are also capable of activating Fcε receptors, e.g. expressed on cells of the immune system, in order to initiate effector functions mediated by IgE.

The epsilon (c) heavy chain is definitive for IgE antibodies, and comprises an N-terminal variable domain VH, and four constant domains Cε1-Cε4. As with other antibody isotypes, the variable domains confer antigen specificity and the constant domains recruit the isotype-specific effector functions.

IgE differs from the more abundant IgG isotypes, in that it is unable to fix complement and does not bind to the Fc receptors FcγRI, RII and RIII expressed on the surfaces of mononuclear cells, NK cells and neutrophils. However, IgE is capable of very specific interactions with the “high affinity” IgE receptor on a variety of immune cells such as mast cells, basophils, monocytes/macrophages, eosinophils (FcεRI, Ka. 10¹¹ M⁻¹), and with the “low affinity” receptor, Fcε RII (Ka. 10⁷ M⁻¹), also known as CD23, expressed on inflammatory and antigen presenting cells (e.g. monocytes/macrophages, platelets, dendritic cells, T and B lymphocytes.

The sites on IgE responsible for these receptor interactions have been mapped to peptide sequences on the Cε chain, and are distinct. The FcεRI site lies in a cleft created by residues between Gln 301 and Arg 376, and includes the junction between the Cε2 and Cε3 domains (Helm, B. et al. (1988) Nature 331, 180183). The FcεRII binding site is located within Cε3 around residue Val 370 (Vercelli, D. et al. (1989) Nature 338, 649-651). A major difference distinguishing the two receptors is that FcεRI binds monomeric Cc, whereas FcεRII will only bind dimerised Cε, i.e. the two Cε chains must be associated. Although IgE is glycosylated in vivo, this is not necessary for its binding to FcεRI and FcεRRII. Binding is in fact marginally stronger in the absence of glycosylation (Vercelli, D. et al. (1989) et. Supra).

Thus binding to Fcε receptors and related effector functions are typically mediated by the heavy chain constant domains of the antibody, in particular by domains which together form the Fc region of the antibody. The antibodies described herein typically comprise at least a portion of an IgE antibody e.g. one or more constant domains derived from an IgE, preferably a human IgE. In particular embodiments, the antibodies comprise one or more domains (derived from IgE) selected from Cε1, Cε2, Cε3 and Cε4. In one embodiment, the antibody comprises at least Cε2 and Cε3, more preferably at least Cε2, Cε3 and Cε4, preferably wherein the domains are derived from a human IgE. In one embodiment, the antibody comprises an epsilon (ε) heavy chain, preferably a human ε heavy chain.

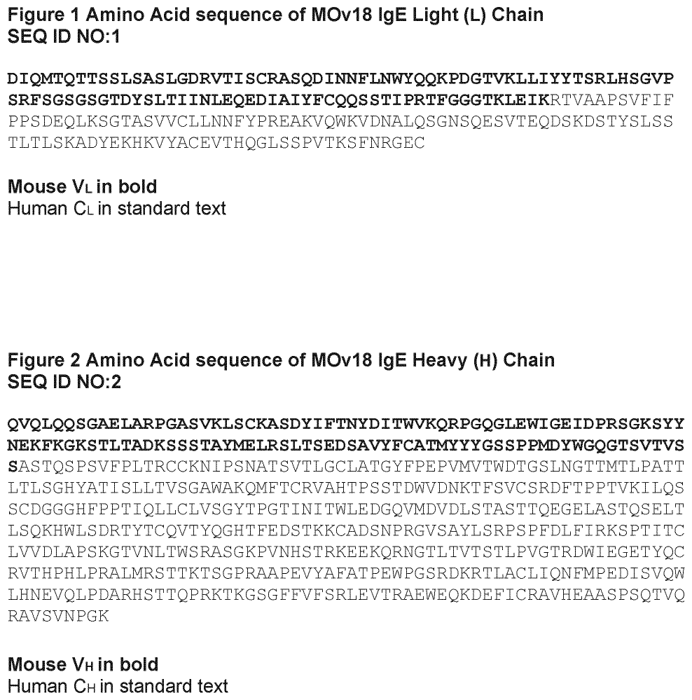

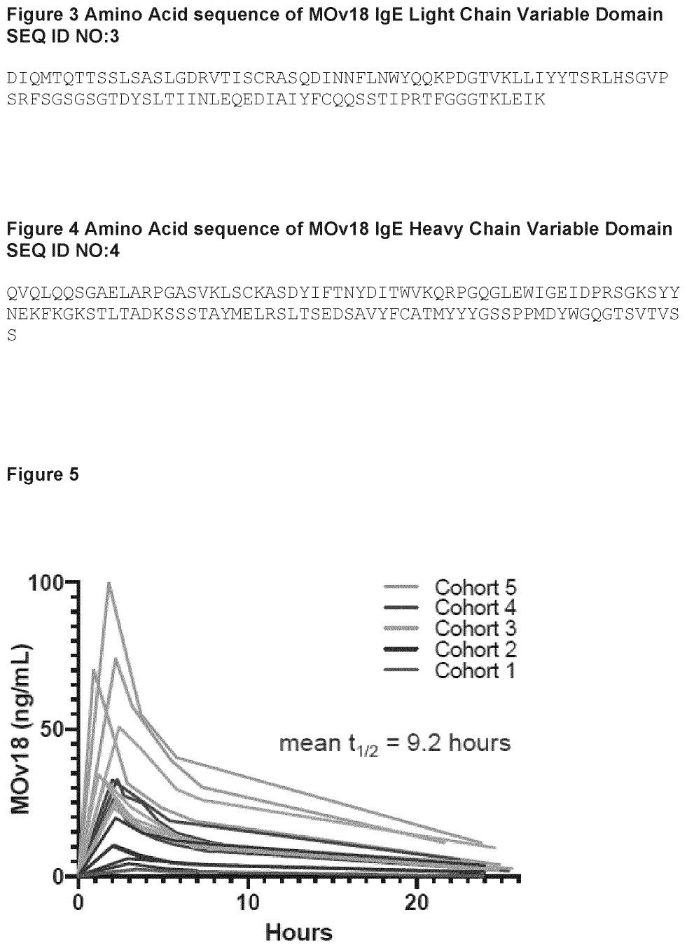

The amino acid sequences of constant domains derived from human IgE are shown in e.g. FIGS. 1 and 2 (SEQ ID NO:s 1 and 2, non-bold text). Nucleotide sequences encoding constant domains derived from human IgE, in particular Cε1, Cε2, Cε3 and Cε4 domains, are also disclosed in e.g. WO 2013/050725. The amino acid sequences of other human and mammalian IgEs and domains thereof, including human Cε1, Cε2, Cε3 and Cε4 domains and human c heavy chain sequences, are known in the art and are available from public-accessible databases. For instance, databases of human immunoglobulin sequences are accessible from the International ImMunoGeneTics Information System (IMGT®) website at http://www.imgt.org. As one example, the sequences of various human IgE heavy (c) chain alleles and their individual constant domains (Cε1-4) are accessible at http://www.imgt.org/IMGT_GENE-DB/GENElect?query=2+IGHE&species=Homo+sapiens.

### Preferred Anti-FRα IgE Antibodies

In one embodiment, the anti-FRα antibody comprises a VH domain comprising at least a portion of the amino acid sequence as defined in SEQ ID NO:4, e.g. comprising at least 20, 30, 50 or 100 amino acids of SEQ ID NO:4 or the full length of SEQ ID NO:4 or one, two or three CDRs present in SEQ ID NO:4 (e.g. defined according to Kabat, Chothia or IMGT).

In one embodiment, the anti-FRα antibody comprises a VL domain comprising at least a portion of the amino acid sequence as defined in SEQ ID NO: 3, e.g. comprising at least 20, 30, 50 or 100 amino acids of SEQ ID NO:3 or the full length of SEQ ID NO:3 or one, two or three CDRs present in SEQ ID NO:3 (e.g. defined according to Kabat, Chothia or IMGT).

In general, functional fragments of the sequences defined above may be used in the present invention. Functional fragments may be of any length as specified above (e.g. at least 50, 100, 300 or 500 nucleotides, or at least 50, 100, 200 or 300 amino acids), provided that the fragment retains the required activity when present in the antibody (e.g. specific binding to FRα and/or a Fcε receptor).

Variants of the above amino acid and nucleotide sequences may also be used in the present invention, provided that the resulting antibody binds an Fcε receptor. Typically such variants have a high degree of sequence identity with one of the sequences specified above.

The similarity between amino acid or nucleotide sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of the amino acid or nucleotide sequence will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of the antibody (e.g. anti-FRα antibody or a domain thereof, e.g. a VL, VH, CL or CH domain) typically have at least about 75%, for example at least about 80%, 90%, 95%, 96%, 97%, 98% or 99% sequence identity with the original sequence (e.g. a sequence defined above), for example counted over the full length alignment with the amino acid sequence of the antibody or domain thereof using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Typically variants may contain one or more conservative amino acid substitutions compared to the original amino acid or nucleic acid sequence. Conservative substitutions are those substitutions that do not substantially affect or decrease the affinity of an antibody to the target antigen (e.g. FRα) and/or Fcε receptors. For example, a human antibody that specifically binds FRα may include up to 1, up to 2, up to 5, up to 10, or up to 15 conservative substitutions compared to the original sequence (e.g. as defined above) and retain specific binding to the FRα polypeptide. The term conservative variation also includes the use of a substituted amino acid in place of an unsubstituted parent amino acid, provided that antibody specifically binds the target antigen (e.g. FRα). Non-conservative substitutions are those that reduce an activity or binding to the target antigen (e.g. FRα) and/or Fcε receptors.

Functionally similar amino acids which may be exchanged by way of conservative substitution are well known to one of ordinary skill in the art. The following six groups are examples of amino acids that are considered to be conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

In some embodiments, the IgE antibody may be conjugated to a cytotoxic moiety, e.g. a chemotherapeutic agent that is capable of (directly) killing cancer cells, in order to produce an antibody-drug conjugate (ADC). The antibody may be conjugated directly to the cytotoxic moiety or via a linker, as is known in the art. For instance, one example of such a cytotoxic agent is maytansinoid DM4, a potent tubulin-targeting agent that is present in the IgG ADC mirvetuximab soravtansine. In other embodiments, the IgE antibody may be administered to a subject in combination with separate (e.g. simultaneous or sequential) administration of a chemotherapeutic agent, e.g gemcitabine (2′, 2′-difluoro 2′deoxycytidine).

However in preferred embodiments, the IgE antibody lacks a cytotoxic moiety and/or is administered as a monotherapy. It has surprisingly been found that anti-FRα IgE antibodies are capable of treating low FRα-expressing tumors without requiring administration of a cytotoxic drug (either in the form of an ADC or in a chemotherapeutic combination).

Thus the antibody may comprise, consist of or consist essentially of (optionally glycosylated) polypeptide chains. For instance the antibody may comprise, consist of or consist essentially of one or more (preferably four) polypeptide chains, e.g. two immunoglobulin heavy chains and optionally two immunoglobulin light chains. Preferably the heavy and/or light chains comprises one or more domains from an IgE antibody.

In particular, it is preferred that the antibody is not an antibody-drug conjugate (ADC). Thus the antibody may lack a further drug or group having a cytotoxic effect, e.g. a chemotherapeutic or drug that is capable of (directly) killing cancer cells. The antibody may further lack a linker or other group for conjugating a cytotoxic moiety to a polypeptide.

### Further IgE Antibodies

As described above, in preferred embodiments the IgE antibody binds to FRα. Preferably the IgE antibodies are capable of inducing cytotoxicity (e.g. ADCC) and/or phagocytosis (ADCP), particularly against cancer cells expressing such an antigen.

In some embodiments, one or more of the variable domains and/or one or more of the CDRs, preferably at least three CDRs, or more preferably all six CDRs may be derived from one or more of the following antibodies: MOv19 (Coney et al., Cancer Res. 1991 Nov. 15; 51(22):6125-32; Coney et al., Cancer Res. 1994 May 1; 54(9):2448-55), mirvetuximab soravtansine (IMGN853, as described in Ab et al., Molecular Cancer Therapeutics 14(7):1605-13, July 2015) or farletuzumab (MORAb-003, as described in Ebel et al., Cancer Immun. 2007; 7:6; Sato and Itamochi, OncoTargets and Therapy 2016:9 1181-1188). Mirvetuximab soravtansine (IMGN853) refers to an immunoconjugate containing the humanized MOv19 (M9346A) antibody, a sulfoSPDB (N-succinimidyl 4-(2-pyridyldithio)-2-sulfobutanoate) linker, and the DM4 maytansinoid (N2′-deacetyl-N2′-(4-mercapto-4-methyl-1-oxopentyl) maytansine).

For instance, the IgE antibody may comprise the following variable domain sequences, or one to six CDRs derived therefrom (e.g. defined according to Kabat, Chothia or IMGT):

Farletuzumab VH domain:
(SEQ ID NO: 7)
EVQLVESGGGVVQPGRSLRLSCSASGFTFSGYGLSWVRQAPGKGL
EWVAMISSGGSYTYYADSVKGRFAISRDNAKNTLFLQMDSLRPED
TGVYFCARHGDDPAWFAYWGQGTPVTVSS
Farletuzumab VL (VK) domain:
(SEQ ID NO: 8)
DIQLTQSPSSLSASVGDRVTITCSVSSSISSNNLHWYQQKPGKAP
KPWIYGTSNLASGVPSRFSGSGSGTDYTFTISSLQPEDIATYYCQ
QWSSYPYMYTFGQGTKVEIK

In other embodiments, the IgE antibody may comprise one or more variable domain sequences or CDRs from an anti-FRα (i.e. anti-FOLR 1) antibody, or antigen-binding fragment, thereof, as defined in e.g. US 2012/0009181 or WO 2018/213260, the contents of which are herein incorporated by reference. For instance, the IgE antibody may comprise the following variable domain sequences, or one to six CDRs derived therefrom (e.g. defined according to Kabat, Chothia or IMGT):

huMOv19 VH domain (SEQ ID NO: 9):
QVQLVQSGAEVVKPGASVKISCKASGYTFTGYFMNWVKQSPGQSL
EWIGRIHPYDGDTFYNQKFQGKATLTVDKSSNTAHMELLSLTSED
FAVYYCTRYDGSRAMDYWGQGTTVTVSS
huMOV19 VL domain (SEQ ID NO: 10):
DIVLTQSPLSLAVSLGQPAIISCKASQSVSFAGTSLMHWYHQKPG
QQPRLLIYRASNLEAGVPDRFSGSGSKTDFTLTISPVEAEDAATY
YCQQSREYPYTFGGGTKLEIKR

For instance, the IgE antibody may comprise one or more (e.g. all six) of the following CDR sequences (defined according to Kabat):

huMOv19 VH-CDR1 (SEQ ID NO: 11):
GYFMN
huMOv19 VH-CDR2 (SEQ ID NO: 12):
RIHPYDGDTFYNQKFQG
huMOv19 VH-CDR3 (SEQ ID NO: 13):
YDGSRAMDY
huMOv19 VL-CDR1 (SEQ ID NO: 14):
KASQSVSFAGTSLMH
huMOv19 VL-CDR2 (SEQ ID NO: 15):
RASNLEA
huMOv19 VL-CDR3 (SEQ ID NO: 16):
QQSREYPYT

In another embodiment, the antibody is a chimaeric, humanized or fully human antibody that specifically binds the epitope bound by mirvetuximab or farletuzumab. The IgE antibody may further comprise one or more IgE constant domains, e.g. Cε1-Cε4 domains, as described above.

### Production of Antibodies and Nucleic Acids

Nucleic acid molecules (also referred to as polynucleotides) encoding the polypeptides provided herein (including, but not limited to antibodies and functional fragments thereof) can readily be produced by one of skill in the art, using the amino acid sequences provided herein, sequences available in the art, and the genetic code. In addition, one of skill can readily construct a variety of clones containing functionally equivalent nucleic acids, such as nucleic acids which differ in sequence but which encode the same effector molecule or antibody sequence. Thus, nucleic acids encoding antibodies are provided herein.

Nucleic acid sequences encoding the antibodies that specifically bind the target antigen (e.g. FRα), or functional fragments thereof, can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence or by polymerization with a DNA polymerase using the single strand as a template. One of skill would recognize that while chemical synthesis of DNA is generally limited to sequences of about 100 bases, longer sequences may be obtained by the ligation of shorter sequences.

Exemplary nucleic acids encoding antibodies, or functional fragments thereof, can be prepared by cloning techniques. Examples of appropriate cloning and sequencing techniques, and instructions sufficient to direct persons of skill through many cloning exercises are found see, for example, Molecular Cloning: A Laboratory Manual, 2nd ed., vol. 1-3, ed. Sambrook et al., Cold Spring Harbor Laboratory Press, Cold Spring Harbor, N.Y., 1989); and Current Protocols in Molecular Biology (Ausubel et al., eds 1995 supplement)). Product information from manufacturers of biological reagents and experimental equipment also provide useful information. Such manufacturers include the SIGMA Chemical Company (Saint Louis, Mo.), R&D Systems (Minneapolis, Minn.), Pharmacia Amersham (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), Invitrogen (Carlsbad, Calif.), and Applied Biosystems (Foster City, Calif.), as well as many other commercial sources known to one of skill.

Nucleic acids encoding native antibodies can be modified to form the antibodies described herein. Modification by site-directed mutagenesis is well known in the art. Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

In one embodiment, antibodies are prepared by inserting a cDNA which encodes one or more antibody domains (e.g. a mouse IgG1 heavy chain variable region which binds human FRα) into a vector which comprises a cDNA encoding one or more further antibody domains (e.g. a human heavy chain c constant region). The insertion is made so that the antibody domains are read in frame that is in one continuous polypeptide which contains a functional antibody region.

In one embodiment, cDNA encoding a heavy chain constant region is ligated to a heavy chain variable region so that the constant region is located at the carboxyl terminus of the antibody. The heavy chain-variable and/or constant regions can subsequently be ligated to a light chain variable and/or constant region of the antibody using disulfide bonds.

Once the nucleic acids encoding the antibody or functional fragment thereof have been isolated and cloned, the desired protein can be expressed in a recombinantly engineered cell such as bacteria, plant, yeast, insect and mammalian cells. It is expected that those of skill in the art are knowledgeable in the numerous expression systems available for expression of proteins including E. coli, other bacterial hosts, yeast, and various higher eukaryotic cells such as the COS, CHO, HeLa and myeloma cell lines.

One or more DNA sequences encoding the antibody or fragment thereof can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art. Hybridomas expressing the antibodies of interest are also encompassed by this disclosure.

The expression of nucleic acids encoding the isolated antibodies and antibody fragments described herein can be achieved by operably linking the DNA or cDNA to a promoter (which is either constitutive or inducible), followed by incorporation into an expression cassette. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signal for introns, maintenance of the correct reading frame of that gene to permit proper translation of mRNA, and stop codons.

To obtain high level expression of a cloned gene, it is desirable to construct expression cassettes which contain, at the minimum, a strong promoter to direct transcription, a ribosome binding site for translational initiation, and a transcription/translation terminator. For E. coli, this includes a promoter such as the T7, trp, lac, or lambda promoters, a ribosome binding site, and preferably a transcription termination signal. For eukaryotic cells, the control sequences can include a promoter and/or an enhancer derived from, for example, an immunoglobulin gene, SV40 or cytomegalovirus, and a polyadenylation sequence, and can further include splice donor and acceptor sequences. The cassettes can be transferred into the chosen host cell by well-known methods such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

When the host is a eukaryote, such methods of transfection of DNA as calcium phosphate coprecipitates, conventional mechanical procedures such as microinjection, electroporation, insertion of a plasmid encased in liposomes, or virus vectors may be used. Eukaryotic cells can also be co-transformed with polynucleotide sequences encoding the antibody, labelled antibody, or functional fragment thereof, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982). One of skill in the art can readily use an expression systems such as plasmids and vectors of use in producing proteins in cells including higher eukaryotic cells such as the COS, CHO, HeLa and myeloma cell lines.

Modifications can be made to a nucleic acid encoding a polypeptide described herein (e.g., a human FRα-specific IgE antibody) without diminishing its biological activity. Some modifications can be made to facilitate the cloning, expression, or incorporation of the targeting molecule into a fusion protein. Such modifications are well known to those of skill in the art and include, for example, termination codons, a methionine added at the amino terminus to provide an initiation, site, additional amino acids placed on either terminus to create conveniently located restriction sites, or additional amino acids (such as poly His) to aid in purification steps. In addition to recombinant methods, the antibodies of the present disclosure can also be constructed in whole or in part using standard peptide synthesis well known in the art.

Once expressed, the recombinant antibodies can be purified according to standard procedures of the art, including ammonium sulfate precipitation, affinity columns, column chromatography, and the like (see, generally, R. Scopes, PROTEIN PURIFICATION, Springer-Verlag, N.Y., 1982). The antibodies, immunoconjugates and effector molecules need not be 100% pure. Once purified, partially or to homogeneity as desired, if to be used therapeutically, the polypeptides should be substantially free of endotoxin.

Often, functional heterologous proteins from E. coli or other bacteria are isolated from inclusion bodies and require solubilization using strong denaturants, and subsequent refolding. During the solubilization step, as is well known in the art, a reducing agent must be present to separate disulfide bonds. An exemplary buffer with a reducing agent is: 0.1 M Tris pH 8, 6 M guanidine, 2 mM EDTA, 0.3 M DTE (dithioerythritol). Reoxidation of the disulfide bonds can occur in the presence of low molecular weight thiol reagents in reduced and oxidized form, as described in Saxena et al., Biochemistry 9: 5015-5021, 1970, and especially as described by Buchner et al., supra.

Renaturation is typically accomplished by dilution (for example, 100-fold) of the denatured and reduced protein into refolding buffer. An exemplary buffer is 0.1 M Tris, pH 8.0, 0.5 M L-arginine, 8 mM oxidized glutathione (GSSG), and 2 mM EDTA.

As a modification to the two chain antibody purification protocol, the heavy and light chain regions are separately solubilized and reduced and then combined in the refolding solution. An exemplary yield is obtained when these two proteins are mixed in a molar ratio such that a 5 fold molar excess of one protein over the other is not exceeded. Excess oxidized glutathione or other oxidizing low molecular weight compounds can be added to the refolding solution after the redox-shuffling is completed.

In addition to recombinant methods, the antibodies, labelled antibodies and functional fragments thereof that are disclosed herein can also be constructed in whole or in part using standard peptide synthesis. Solid phase synthesis of the polypeptides of less than about 50 amino acids in length can be accomplished by attaching the C-terminal amino acid of the sequence to an insoluble support followed by sequential addition of the remaining amino acids in the sequence. Techniques for solid phase synthesis are described by Barany & Merrifield, The Peptides: Analysis, Synthesis, Biology. Vol. 2: Special Methods in Peptide Synthesis, Part A. pp. 3-284; Merrifield et al., J. Am. Chem. Soc. 85:2149-2156, 1963, and Stewart et al., Solid Phase Peptide Synthesis, 2nd ed., Pierce Chem. Co., Rockford, Ill., 1984. Proteins of greater length may be synthesized by condensation of the amino and carboxyl termini of shorter fragments.

Methods of forming peptide bonds by activation of a carboxyl terminal end (such as by the use of the coupling reagent N,N′-dicylohexylcarbodimide) are well known in the art.

In one embodiment, the antibodies, nucleic acids, expression vectors, host cells or other biological products are isolated. By “isolated” it is meant that the product has been substantially separated or purified away from other biological components in the environment (such as a cell) in which the component naturally occurs, i.e., other chromosomal and extra-chromosomal DNA and RNA, proteins and organelles. Nucleic acids and antibodies that have been “isolated” include nucleic acids and antibodies purified by standard purification methods. The term also embraces nucleic acids and antibodies prepared by recombinant expression in a host cell as well as chemically synthesized nucleic acids.

### Compositions and Therapeutic Methods

Compositions are provided herein that include a carrier and one or more therapeutic IgE antibodies, or functional fragments thereof. The compositions can be prepared in unit dosage forms for administration to a subject. The antibody can be formulated for systemic or local (such as intra-tumour) administration. In one example, the therapeutic IgE antibody is formulated for parenteral administration, such as intravenous administration or subcutaneous administration.

The compositions for administration can include a solution of the antibody (or a functional fragment thereof) dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of the antibody and excipients in these formulations can vary, and will be selected primarily based on fluid volumes, viscosities, body weight and the like in accordance with the particular mode of administration selected and the subject's needs. Actual methods for preparing administrable compositions will be known or apparent to those skilled in the art and are described in more detail in such publications as Remington's Pharmaceutical Science, 19th ed., Mack Publishing Company, Easton, Pa. (1995).

In preferred embodiments the compositions are provided as unit dosage forms, e.g. comprising a defined amount of the IgE antibody suitable for administration to a subject in a single dose. The unit dosage forms may be packaged individually, e.g. in single containers, vials, pre-filled syringes or the like. The unit dosage forms may be suitable for immediate administration to the subject (e.g. may comprise a physiologically acceptable concentration of salts) or the unit dosage forms may be provided in concentrated or lyophilized form (e.g. for dilution with sterile saline solution before use).

The anti-FRα IgE antibody may be administered at any suitable dose. However in preferred embodiments described herein, a typical unit dose of the pharmaceutical composition (e.g. for intravenous administration) comprises less than 50 mg of the IgE antibody. For instance, the composition (i.e. in unit dosage form) may comprise less than 40 mg, 30 mg, 25 mg, 20 mg, 15 mg, 10 mg, 5 mg, 3 mg, or 1 mg of the IgE antibody. The composition may comprise at least 10 μg, 100 μg, 200 μg, 300 μg, 500 μg, 700 μg, 1 mg, 3 mg, 5 mg or 10 mg of the IgE antibody. In preferred embodiments, the composition comprises comprising 10 μg to 50 mg, 70 μg to 30 mg, 300 μg to 50 mg, 300 μg to 30 mg, 300 μg to 3 mg, 500 μg to 50 mg, 500 μg to 30 mg, 500 μg to 10 mg, 500 μg to 3 mg, 700 μg to 50 mg, 700 μg to 30 mg, 700 μg to 10 mg, 700 μg to 3 mg, 500 μg to 5 mg, 500 μg to 1 mg, or about 700 μg of the IgE antibody. In some embodiments, the composition may comprise an amount of the IgE antibody within one or more of the above ranges, but excluding one or more of the following amounts: 1p g, 5p g, 10 μg, 50 μg, 100 μg, 500 μg, 1 mg, 2 mg, 4 mg, 5 mg, 10 mg or 15 mg. For instance, the composition may comprise 2 μg to 9 μg, 11 μg to 99 μg, 101 μg to 499 μg, 501 to 999 μg or 2 mg to 9 mg.

The dosage of the IgE antibody administered to the subject may be based on the subject's body weight. Thus the dose of the IgE antibody administered to the subject may be e.g. less than 1 mg/kg. Preferably the IgE antibody may be administered to the subject in a dose (per administration) of e.g. less than 0.7 mg/kg, 0.5 mg/kg, 0.3 mg/kg, 0.1 mg/kg, 0.07 mg/kg, 0.05 mg/kg, 0.03 mg/kg or 0.01 mg/kg. The dose of the IgE antibody administered to the subject may be at least 0.001 mg/kg, 0.003 mg/kg, 0.005 mg/kg, 0.007 mg/kg, 0.01 mg/kg, 0.05 mg/kg or 0.1 mg/kg. In preferred embodiments, the dose of the IgE antibody administered to the subject may be 0.001-1 mg/kg, 0.003-0.7 mg/kg, 0.005-0.5 mg/kg, 0.005-0.1 mg/kg, 0.005-0.05 mg/kg, 0.007-0.03 mg/kg or 0.007-0.15 mg/kg. In some embodiments, the dose of the IgE antibody administered to the subject may be within one or more of the above defined ranges, but excluding one or more of the following dosages: 1p g/kg, 10 μg/kg, 100 μg/kg or 0.5 mg/kg. For instance, the dose of the IgE antibody may be 2 to 9 μg/kg, 11 to 99 μg/kg, 101 to 499 μg/kg or 0.51 to 0.7 mg/kg.

In embodiments of the present invention, the unit dosages of the IgE antibody described above are at administered at most once a week, e.g. the maximum weekly dose of the IgE antibody is 50 mg, 40 mg, 30 mg, 25 mg, 20 mg, 15 mg, 10 mg, 5 mg, 3 mg, or 1 mg. For instance the weekly dose of the IgE antibody may be 10 μg to 50 mg, 70 μg to 30 mg, 300 μg to 50 mg, 300 μg to 30 mg, 300 μg to 3 mg, 500 μg to 50 mg, 500 μg to 30 mg, 500 μg to 10 mg, 500 μg to 3 mg, 700 μg to 50 mg, 700 μg to 30 mg, 700 μg to 10 mg, 700 μg to 3 mg, 500 μg to 5 mg, 500 μg to 1 mg, or about 700 μg. The weekly dose of the IgE antibody may also be determined according to the subject's body weight, e.g. the IgE antibody may be administered to the subject in a dose of e.g. less than 0.7 mg/kg/week, 0.5 mg/kg/week, 0.3 mg/kg/week, 0.1 mg/kg/week, 0.07 mg/kg/week, 0.05 mg/kg/week, 0.03 mg/kg/week or 0.01 mg/kg/week. In preferred embodiments, the dose of the IgE antibody administered to the subject may be 0.001-1 mg/kg/week, 0.003-0.7 mg/kg/week, 0.005-0.5 mg/kg/week, 0.005-0.1 mg/kg/week, 0.005-0.05 mg/kg/week, 0.007-0.03 mg/kg/week or 0.007-0.15 mg/kg/week. In some embodiments, the dose of the IgE antibody administered to the subject may be within one or more of the above defined ranges, but excluding one or more of the following dosages: 1 μg/kg/day (7 μg/kg/week), 10 μg/kg/day (70 μg/kg/week) or 100 μg/kg/day (0.7 mg/kg/week). For instance, the dose of the IgE antibody may be 2 to 6 μg/kg/week, 8 to 69 μg/kg/week, or 71 to 699 μg/kg/week.

In one embodiment, the pharmaceutical composition is a liquid comprising one or more excipients selected from sodium citrate, L-arginine, sucrose, polysorbate 20 and/or sodium chloride. Preferably the composition has a pH of 6.0 to 8.0, e.g. about 6.5. Preferred concentrations of the excipients include: 0.05 to 0.5 M (e.g. about 0.1 M) sodium citrate; 10 to 50 g/L (e.g. about 30 g/L) L-arginine; 10 to 100 g/L (e.g. about 50 g/L) sucrose; 0.01 to 0.05% w/w (e.g. 0.02% w/w) polysorbate 20. In one embodiment, the IgE antibody is present in such a formulation at a concentration of about 0.1 mg/ml to 10 mg/ml or 0.5 mg/ml to 2 mg/ml, e.g. about 1 mg/ml. In some embodiments, such a composition may be formulated as a unit dosage form e.g. in a volume of about 1 ml of solution comprising about 1 mg of the IgE antibody, for instance in a 2 ml type I glass vial. The composition may be diluted with sterile saline (0.9% w/v) before administration to the subject, e.g. in an amount of 1 ml of the composition in 250 ml of saline.

Antibodies may be provided in lyophilized form and rehydrated with sterile water before administration, although they are also provided in sterile solutions of known concentration. The antibody solution is then added to an infusion bag containing 0.9% sodium chloride, USP, and administered to the subject. Considerable experience is available in the art in the administration of antibody drugs, which have been marketed in the U.S. since the approval of RITUXAN (Registered trademark) in 1997. Antibodies can be administered by slow infusion, rather than in an intravenous push or bolus. In one example, a higher loading dose is administered, with subsequent, maintenance doses being administered at a lower level. For example, an initial loading dose may be infused over a period of some 90 minutes, followed by weekly maintenance doses for 4-8 weeks infused over a 30 minute period if the previous dose was well tolerated.

The antibody (or functional fragment thereof) can be administered to slow or inhibit the growth of cells, such as cancer cells. In these applications, a therapeutically effective amount of an antibody is administered to a subject in an amount sufficient to inhibit growth, replication or metastasis of cancer cells, or to inhibit a sign or a symptom of the cancer. In some embodiments, the antibodies are administered to a subject to inhibit or prevent the development of metastasis, or to decrease the size or number of metasases, such as micrometastases, for example micrometastases to the regional lymph nodes (Goto et al., Clin. Cancer Res. 14(11):3401-3407, 2008).

Thus in some embodiments, the IgE antibody is used to treat cancer and/or to delay or prevent the progression of cancer. By “delay or prevent the progression” of cancer it is meant that, for example, the cancer is at least stable for a period of time after administration of the antibody, e.g. for at least 6 weeks, at least 12 weeks, at least 6 months or at least 12 months. “Stable” disease may be defined e.g. as a change in the RECIST score of less than 20%.

RECIST (Response Evaluation Criteria in Solid Tumours) evaluation is a simple method for determining whether a patient's disease has improved, stayed about the same, or worsened following treatment with a cancer therapeutic, and is commonly used in clinical trials of anticancer agents. The RECIST criteria are specified e.g. in Eisenhauer et al., New response evaluation criteria in solid tumours: Revised RECIST guideline (version 1.1), European Journal Of Cancer 45 (2009) 228-247. RECIST defines Progressive Disease (PD) as at least a 20% increase in the sum of diameters of target lesions, taking as reference the smallest sum on study. Stable disease is defined as neither sufficient shrinkage to qualify for Partial Response (at least a 30% decrease in the sum of diameters of target lesions), nor sufficient increase to qualify for PD, i.e. an increase of <20% is defined as stable disease.

It will be appreciated that in this context “at least stable” includes an increase or decrease in the RECIST score of less than 20%. Thus the antibody may delay or prevent progression of the disease (e.g. delay or prevent appearance of one or more signs or symptoms or cancer, and/or inhibit the growth of cancer cells and/or prevent or reduce metastases), or ameliorate or promote remission of the disease (e.g. reduce or inhibit one or more sign or symptom of cancer, and/or kill cancer cells).

### Subjects

Suitable subjects may include those diagnosed with cancer, e.g. a cancer that expresses FRα, such as, but not limited to, skin cancer (e.g. melanoma), lung cancer, prostate cancer, squamous cell carcinoma (such as head and neck squamous cell carcinoma), breast cancer (including, but not limited to basal breast carcinoma, ductal carcinoma and lobular breast carcinoma), leukemia (such as acute myelogenous leukemia and 11g23-positive acute leukemia), lymphoma, a neural crest tumour (such as an astrocytoma, glioma or neuroblastoma), ovarian cancer, colon cancer, stomach cancer, pancreatic cancer, bone cancer (such as a chordoma), glioma or a sarcoma (such as chondrosarcoma). Preferably the antibody is administered to treat a solid tumour. Preferably the subject is human.

A therapeutically effective amount of antibody will depend upon the severity of the disease and the general state of the patient's health. A therapeutically effective amount of the antibody is that which provides either subjective relief of a symptom(s) or an objectively identifiable improvement as noted by the clinician or other qualified observer. These compositions can be administered in conjunction with another chemotherapeutic agent, either simultaneously or sequentially.

The anti-FRα antibody is used to treat a subject suffering from a low FRα expressing tumor. Such subjects may be referred to as “low FRα expressors”, in contrast to moderate or high FRα expressors. The terms “low FRα-expressing tumor” and “low FRα expressor” are well understood in the field of cancer therapy and are frequently used to describe specific patient groups (see e.g. Cristea et al., Journal of Clinical Oncology 2021 39:15_suppl, 5542; Martin et al. Gynecol Oncol. 2017 November; 147(2): 402-407). Thus the subgroup of patients having low tumor FRα expression is clearly distinguished from high FRα expressors (see e.g. SORAYA trial, ClinicalTrials.gov Identifier: NCT04296890).

### Detecting Low FRα Expression

Low tumor FRα expression may be determined using well known and standard techniques. Typically FRα expression is determined in a biopsy sample from the tumor. Techniques for obtaining biopsy samples from tumor tissue are known in the art, as are histopathological techniques for processing such samples. For instance, biopsy tissue samples may be fixed with formalin and embedded in paraffin or processed fresh before sectioning and placement on microscope slides for light microscopy analysis and imaging.

Haemotoxylin and eosin (H&E) staining of paraffin-embedded sections is the default technology to visualize tissue on a glass slide for pathology analysis. Immunohistochemistry (IHC) staining is a well-known approach to identify expression of proteins on cells in pathology tissue slides. The staining results in a typical brown appearance of tissue where the targeted protein is overexpressed as compared to normal. For example, by using an antibody against FRα, its expression levels can be detected.

Suitable techniques for detecting FRα expression in tumor samples are described in, for example, Zhao et al., “Development and Application of An Immunohistochemistry-based Clinical Assay for Evaluating Folate Receptor Alpha (FRα) Expression in the Clinical Setting”, Abstract 3400A, AACR annual meeting, Apr. 18-22, 2015; Ab et al. (2015), Molecular Cancer Therapeutics 14(7):1605-13; Altwerger et al., Mol Cancer Ther. 2018 May; 17(5): 1003-1011 and Martin et al. “Gynecol Oncol. 2017 November; 147(2): 402-407. For instance, FRα expression may be determined using a Ventana FOLR1 (FOLR-2.1) CDx assay, i.e. cells are stained with the antibody FOLR1-2.1 using Ventana Medical System's Discovery Ultra instrument (see above citations and ClinicalTrials.gov Identifier: NCT04296890).

Any suitable anti-FRα antibody may be used in such methods, e.g. an anti-FRα IgG antibody (polyclonal or monoclonal). Various anti-FRα antibodies suitable for use in immunohistochemistry are available from commercial sources, e.g. Thermofisher/Invitrogen (cat. no. PA5-42004), Leica Biosystems (e.g. BN3.2), Enzo Life Sciences (e.g. clone 548908, cat. no. ENZ-ABS378-0100), Abcam (e.g. ab67422) and Immunogen (353.2.1, FOLR1-2.1). Preferably the anti-FRα antibody used for detection of FRα is BN3.2, e.g. as described in Smith et al., “A novel monoclonal antibody for detection of folate receptor alpha in paraffin-embedded tissues”, Hybridoma (Larchmt). 2007 October; 26(5):281-8, doi: 10.1089/hyb.2007.0512.

By “low FRα-expressing tumor” it is typically meant that less than 50% of tumor cells in the subject express FRα. Preferably less than 40%, 30%, 25%, 20% or 10% of tumor cells in the subject express FRα. These levels of expression typically refer to membrane FRα expression that is detectable by immunohistochemistry, e.g. using the methods described above.

As described in the publications cited above, FRα expression may be classified according to both the percentage of cells showing membrane FRα expression and the FRα staining intensity in the tissue sample. In some cases, FRα staining intensity may be classified on a scale from 0 (no detectable FRα above isotype control) to 3 (high/strong FRα staining intensity). Typically 1 indicates low/weak FRα staining intensity and 2 indicates moderate FRα staining intensity. Classification according to FRα staining intensity can also be combined with a determination of the percentage of tumor cells positive for FRα, (e.g. from 0 to 100%).

For instance, in some cases less than 50%, 40%, 30%, 25%, 20% or 10% of tumor cells in the subject show moderate- or high- (2+) intensity staining for membrane FRα. For instance, in one embodiment less than 25% of tumor cells show moderate- or high- (i.e. 2+) intensity staining for membrane FRα. Thus in one embodiment, the FRα expression level may be classified using a “PS2+” scoring method, e.g. as described in Martin et al. Gynecol Oncol. 2017 November; 147(2): 402-407; Cristea et al., Journal of Clinical Oncology 2021 39:15_suppl, 5542; ClinicalTrials.gov Identifier: NCT02996825; and Immunogen press release dated Sep. 29, 2019, ImmunoGen Presents Full Data from Phase 3 FORWARD I Study of Mirvetuximab Soravtansine in Ovarian Cancer at ESMO, available at https://investor.immunogen.com/news-releases/news-release-details/immunogen-presents-full-data-phase-3-forward-i-study or WO 2018/213260).

Preferably an overall tumor FRα expression score for the subject is determined. The overall tumor FRα expression score may be defined as the product of the membrane FRα staining intensity score (i.e. 0 to 3) for the subject and the percentage of tumor cells positive (e.g. at least 1+) for FRα (0 to 100). This gives a maximum overall score of 300. An overall score of 201 to 300 may be defined as high expression, 101 to 200 as moderate expression and 100 or below as low expression. Thus in some embodiments the overall tumor FRα expression score for the subject may be 100 or less, preferably less than 90, 80, 70, 60, 50 or 40 or less, more preferably 30 or less or 20 or less.

In other embodiments, an “H-score” is calculated e.g. as described in Altwerger et al., Mol Cancer Ther. 2018 May; 17(5): 1003-1011. The H-score is calculated by determining the level of FRα expression (i.e., intensity level of membrane staining on each cell (0-3, 0=negative, 1=weak, 2=moderate and 3=strong), and the percentage of cells in a representative field at each staining intensity (0-100%) as follows: [1×(% cells 1+)+2×(% cells 2+)+3×(% cells 3+)]. This gives a final score ranging from 0 to 300. An H score of 201 to 300 may be defined as high expression, 101 to 200 as moderate expression and 100 or below as low expression. Thus in some embodiments the H score for the subject may be 100 or less, preferably less than 90, 80, 70, 60, 50 or 40 or less, more preferably 30 or less or 20 or less.

In alternative embodiments, tumor FRα expression in the subject may be compared to that in a population of cancer subjects. Typically the population of cancer subjects refers to other subjects suffering from the same type of cancer. Thus the relative level of tumor FRα expression in the subject, compared to other subjects with the same type of cancer, may be ascertained. Since this method does not require an absolute quantitation of FRα expression levels, but only a relative determination compared to other cancer subjects, any systematic bias due to lack of standardization of expression detection is avoided.

In preferred embodiments, tumor (e.g. membrane) FRα expression in the subject is lower than in at least 50% of cancer subjects. Preferably membrane FRα expression in tumor cells of the subject is lower than in at least 60%, at least 70% or at least 80% of cancer subjects, e.g. in subjects suffering from the same form of cancer (e.g. ovarian cancer). More preferably tumor membrane FRα expression in the subject is lower than in at least 50%, 60%, 70%, 80% or at least 90% of FRα-expressing tumors (e.g. in FRα-expressing ovarian tumors).

It is preferred that the tumor expresses FRα, i.e. the tumor shows at least some FRα expression. Typically this means that tumor cells in the subject show detectable membrane FRα expression, e.g. by immunohistochemistry. More preferably at least 1% or at least 5% of tumor cells in the subject show detectable (e.g. membrane) FRα expression, e.g. using immunohistochemical detection of FRα. In other embodiments at least 10%, 15% or 20% of tumor cells in the subject show detectable membrane FRα expression.

Preferably the determination of FRα expression is made on a recent tumor biopsy sample, i.e. a biopsy sample obtained from the subject shortly before the start of anti-FRα IgE treatment (rather than an old or archived sample). For instance, the biopsy sample may be obtained and/or FRα expression determined less than 6 months, 3 months, 1 month, 2 weeks, 1 week, 3 days, 48 hours or 24 hours before the start of anti-FRα IgE treatment.

The invention will now be further described by way of example only, with reference to the following non-limiting embodiments.

### Example

### Chimeric MOv8 IgE (MOv08 IgE)

Chimeric MOv18 IgE (MOv18 IgE) is an anti-folate receptor α (FRα) monoclonal antibody (mAb) of the IgE class. This antibody has been shown to mediate potent antibody-dependent cell-mediated cytotoxicity (ADCC) and antibody-dependent cell mediated phagocytosis (ADCP) against FRα expressing tumour cell lines in vitro and FRα expressing tumour xenografts in vivo. The target antigen, FRα, is over-expressed in several solid cancer types (including ovarian and endometrial cancer and mesothelioma). The antigen has been characterised as effectively tumour specific and clinical trials targeting FRα using IgG antibodies, including trials of chimeric MOv18 IgG1 (MOv18 IgG1), have demonstrated favourable tolerability profiles. MOv18 IgE is thought to have a mechanism of action involving binding to blood effector cells including monocytes, basophils and eosinophils. These IgE carrying cells enter tissues and, upon encountering FRα expressing cells, produce an IgE mediated immune response against the tumour. The MOv18 IgG and IgE antibodies and their properties are described, for example, in Coney, L. R., A. Tomassetti, et al. (1991). “Cloning of a tumor-associated antigen: MOv18 and MOv19 antibodies recognize a folate-binding protein.” Cancer Res 51(22): 6125-6132; Gould, H. J., G. A. Mackay, et al. (1999). “Comparison of IgE and IgG antibody-dependent cytotoxicity in vitro and in a SCID mouse xenograft model of ovarian carcinoma.” Eur J Immunol 29(11): 3527-3537; Karagiannis, S. N., Q. Wang, et al. (2003). “Activity of human monocytes in IgE antibody dependent surveillance and killing of ovarian tumor cells.” Eur J Immunol 33(4): 1030-1040.

### Drug Formulation and Administration

MOv18 IgE is a 168 kDa protein having light and heavy chain amino acid sequences as shown in FIGS. 1 (SEQ ID NO:1) and 2 (SEQ ID NO:2). The MOv18 IgE drug product is supplied as a sterile, pyrogen-free and particle-free solution containing 1 mg/mL MOv18 IgE at pH 6.5 as a 1.0 mL fill in a 2 mL vial for dilution prior to infusion. Each vial of MOv18 IgE also contains the excipients 0.1 M sodium citrate, 30 g/L L-arginine 50 g/L sucrose, 0.02% polysorbate 20 in water for injection.

MOv18 IgE is administered by intravenous (IV) infusion after dilution with 0.9% (w/v) saline. In addition, subjects received intradermal [ID] administration of MOv18 IgE (plus histamine and saline controls), prior to every IV administration, to evaluate the risk of anaphylaxis. Alternatively, skin prick testing was used to evaluate anaphylaxis risk. Patients only proceeded to IV administration of MOv18 IgE in the absence of a cutaneous reaction to the antibody.

Prior to administration of the therapeutic antibody, blood samples were obtained from the subjects and subjected to a basophil activation test in the presence of the MOv18 IgE (Flow CAST®, Bühlmann Laboratories AG, Schönenbuch, Switzerland).

### Study Design

MOv18 IgE was tested in open label, Phase I multiple ascending dose escalation trial in FRα positive solid tumours. 24 patients with advanced solid tumours expressing FRα were entered into the study. Eligible subjects had adequate organ function, no history of severe allergy and an absence of concomitant medications or comorbidities that could increase risk in the event of anaphylaxis.

FRα expression in each patient was determined in tumor biopsy samples using an anti-FRα BN3.2 primary antibody (available from Leica Biosystems) and methodology as described by Lawson and Scorer (Lawson, N. and P. Scorer (2010). “Evaluation of Antibody to Folate Receptor-alpha (FR-α).” 16(21): 5288-5295, available from http://www.leicabiosystems.com/pathologyleaders/evaluation-of-antibody-to-folate-receptoralpha-fr-% CE % B1/.) The method for determining FRα expression is also described in e.g. Ab et al., “IMGN853, a Folate Receptor-α (FRα)-Targeting Antibody-Drug Conjugate, Exhibits Potent Targeted Antitumor Activity against FRα-Expressing Tumors”, Molecular Cancer Therapeutics 14(7):1605-13, July 2015. Immunohistochemical (IHC) staining of FRα was performed in formalin-fixed paraffin-embedded tissue from tumor biopsy samples on microscope slides. Slides were baked, dewaxed and treated with hydrogen peroxide to inactivate endogenous peroxidase. Slides were incubated with either the anti-FRα murine monoclonal antibody BN3.2 or an isotype control, specific staining visualized and counterstained with hematoxylin. All tissue slides were evaluated and scored by a qualified pathologist. FRα staining intensity was scored on a scale of 0 to 3 (0=no specific staining similar to that of the isotype control, 1=weak, 2=moderate, and 3=strong staining). The percentage of tumor cells showing FRα expression (i.e. at least 1+ staining intensity) was also determined. Subjects in this study showed expression of FRα (i.e. 1+, 2+ or 3+ membrane staining) on at least 5% of tumour cells by immunohistochemistry.

Patients received MOv18 IgE once weekly as an IV infusion for a total of six doses, starting at a flat dose of 70 μg. Dose escalation was carried out through defined dose levels (including 70 μg, 250 μg, 500 μg, 700 μg, 1.5 mg and 3 mg) up to a maximum dose of 50 mg. Three weeks of treatment were considered as one cycle during this phase. Patients in any cohort who appeared to be benefitting from MOv18 IgE were given up to three further doses of MOv18 IgE at fortnightly intervals continuing at the same dose level (unless dose-limiting toxicity was encountered or the patient developed progressive disease). This additional period was considered a maintenance phase.

Initially, patients were enrolled in single patient cohorts (Cohorts 1 to 4; 70 to 700 μg doses of MOv18 IgE), as the planned doses are very low and were considered unlikely to provoke a significant biological response. For Cohorts 5 to 10 (1.5 to 50 mg doses of MOv18 IgE), three patients were enrolled per cohort, with an additional three patients added to a cohort if needed for toxicity. Cohorts were expanded up to six patients to further explore the safety and efficacy of MOv18 IgE. No further dose escalation was carried after Cohort 10 (i.e., 50 mg was the top dose evaluated).

### Results

In an initial phase of the study, 10 patients had received MOv18 IgE with two patients treated at the 500 μg dose level. Anti-drug antibodies were detected in 3/21 evaluable patients (ADA detected in 2 patients, plus one patient with suspected ADA) at 6 weeks and/or at off study follow up (>8 weeks). One of the patients treated at the 500 μg dose level experienced a grade 3 anaphylactic episode shortly after receiving MOv18 IgE. The patient responded to standard anaphylaxis treatment as per protocol and recovered fully.

The pharmacokinetics of MOv18 IgE (serum concentration) following intravenous administration for particular dose cohorts of subjects are shown in FIG. 5. Cohort 1: 70 μg; Cohort 2: 250 μg; Cohort 3: 500 μg; Cohort 4: 700 μg; Cohort 5: 1.5 mg.

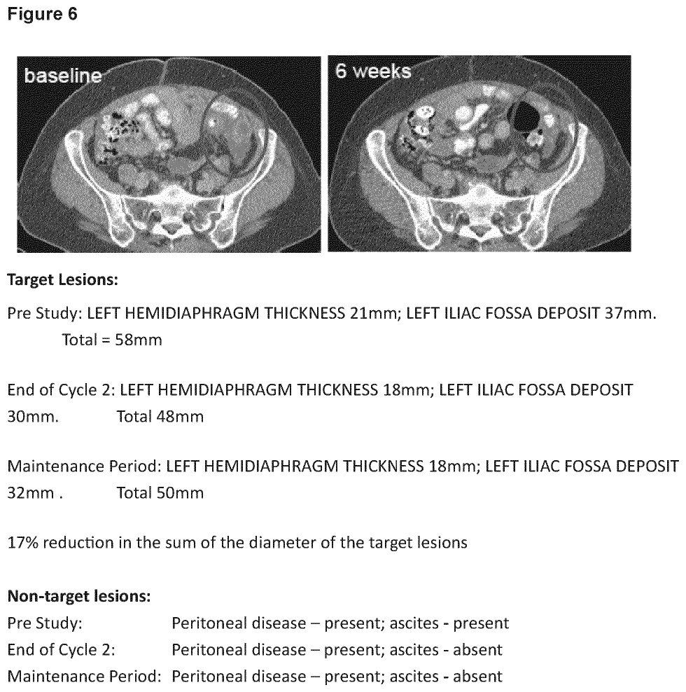

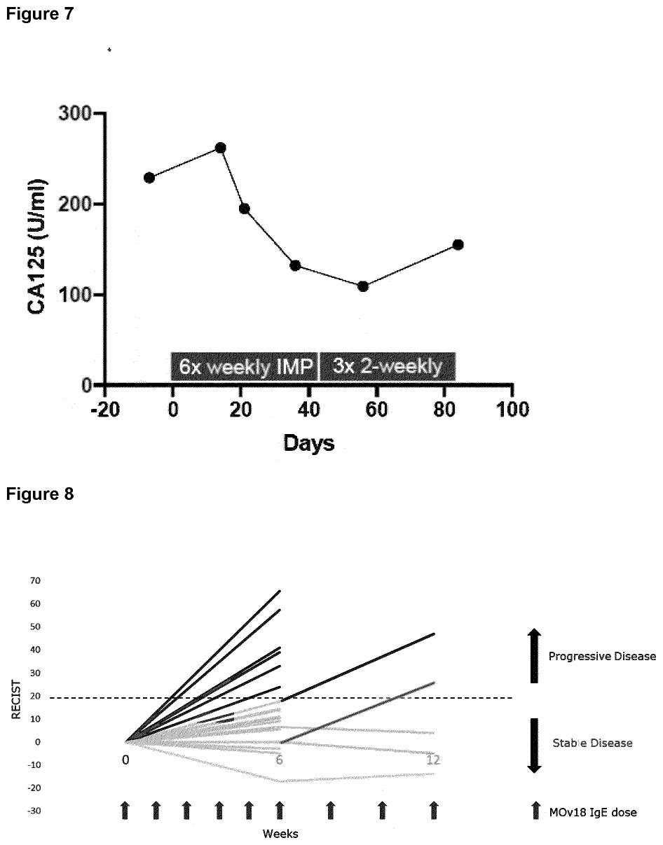

FIGS. 6 and 7 show that administration of MOv18 IgE antibody at a unit dose of 700 μg resulted in anti-tumor effects. FIG. 6 shows the results of tumor measurements taken from a CT scan image showing a reduction in tumor size in an ovarian cancer subject treated with the 700 μg dose level of MOv18 IgE antibody. FIG. 7 shows a significant decrease in serum concentration of the ovarian cancer antigen CA125 during treatment of a patient with 6 weekly doses of 700 μg MOv18 IgE antibody, followed by 3 further 700 μg doses of the antibody at 2 week intervals. A reduction in CA125 during ovarian cancer treatment has been demonstrated to be associated with positive treatment outcomes (see e.g. Yang, Z., Zhao, B. & Li, L. The significance of the change pattern of serum CA125 level for judging prognosis and diagnosing recurrences of epithelial ovarian cancer. J Ovarian Res 9, 57 (2016)). The decrease in CA125 levels shown in FIG. 7 is above the threshold for defining a response to chemotherapy in ovarian cancer according to the Gynecologic Oncology Group (GOG) criteria (see e.g. Rustin et al. Defining response of ovarian carcinoma to initial chemotherapy according to serum CA 125, Journal of Clinical Oncology 1996 14:5, 1545-1551).

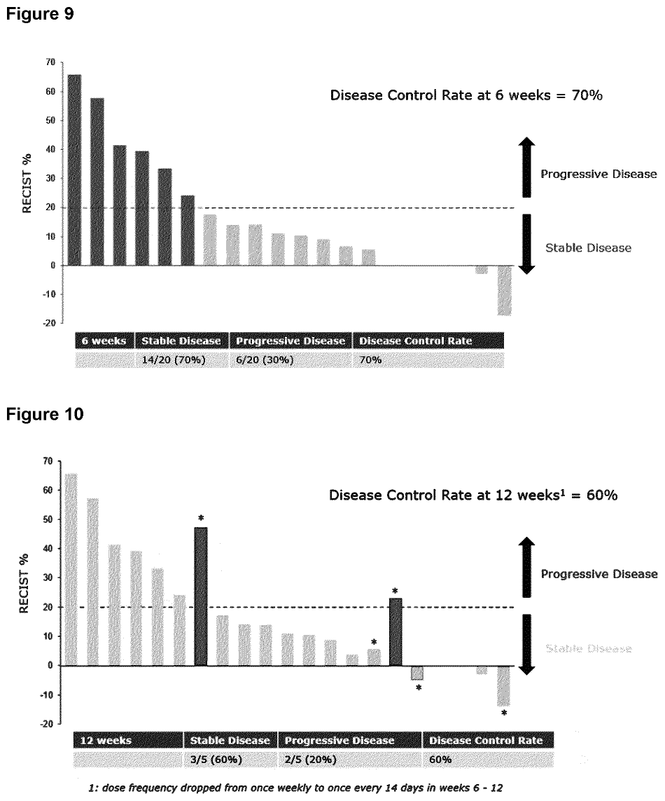

FIGS. 8 to 10 show that a majority of patients treated with low doses of the MOv18 IgE antibody experienced stable disease. FIG. 8 is a plot of the change in RECIST (Response Evaluation Criteria in Solid Tumours) scores in individual ovarian cancer subjects treated with the MOv18 IgE antibody from the start to end of treatment (0-12 weeks). FIGS. 9 and 10 show the change in RECIST scores in individual subjects at 6 and 12 weeks treatment respectively. A change in RECIST score of less than 20% is indicative of stable disease. After 6 weeks treatment, 70% (14/20) of patients treated had stable disease. 60% (3/5) of patients treated to 12 weeks still had stable disease. Not that the dose frequency dropped from once weekly to once every two weeks in period between 6-12 weeks.

Stable disease (i.e. a change in RECIST score of less than 20%) is a key driver of Progression Free Survival (PFS), an efficacy primary endpoint. The present study indicates a Disease Control Rate of 70% at 6 weeks and 60% at 12 weeks. Thus these results demonstrate that an IgE antibody can be used in humans at a very low dose to treat and/or delay progression of cancer in a subject.

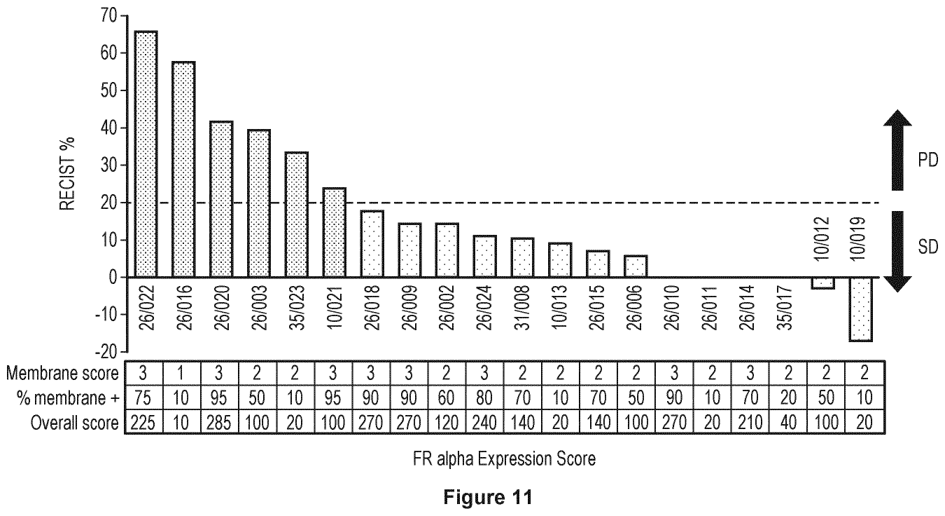

FIG. 11 shows that MOv18 IgE was effective in treating subjects with low FRα antigen levels. The inclusion criteria for this study required that at least 5% of tumour cells were positive for FRα by immunohistochemistry. However, of subjects showing stable disease at 6 weeks, 6/14 were low FRα expressors, indicated by an overall FRα expression score of 100 or less. In each of these subjects, 50% or less of tumour cells were positive for FRα.

Of 6 subjects showing no change or a decrease in RECIST score at 6 weeks, 4 were low FRα expressors (overall FRα expression score of 100 or less, and 50% or fewer of FRα-positive tumor cells). Moreover the two best responders in the study (both of whom showed a decrease in RECIST score) were both low FRα expressors. The subject showing the best response (as shown in FIGS. 6 and 7) had a very low overall FRα expression score of 20, and only 10% of FRα-positive tumour cells.

The results show that MOv18 IgE is well suited for anti-cancer therapy and that administration is tolerable in most patients. Most strikingly, the antibody shows anti-tumor activity even at very low doses (e.g. 700 μg) in low FRα expressing subjects. These results support for the first time the safety and efficacy of IgE as a treatment for low FRα-expressing tumors, including at unit doses of less than 50 mg (less than 1 mg/kg/week).

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. An anti-folate receptor alpha (FRα) immunoglobulin E (IgE) antibody for use in treating a low FRα-expressing tumor in a subject.

2. An IgE antibody for use according to claim 1, wherein less than 50% of tumor cells in the subject express FRα; preferably wherein less than 40%, 30%, 20% or 10% of tumor cells in the subject express FRα.

3. An IgE antibody for use according to claim 1 or claim 2, wherein less than 50%, 30% or 20% of tumor cells in the subject show detectable membrane FRα expression using immunohistochemical detection of FRα.

4. An IgE antibody for use according to any preceding claim, wherein using immunohistochemical detection of FRα, less than 50%, 30% or 20% of tumor cells in the subject show moderate- or high- (2+) intensity staining for membrane FRα.

5. An IgE antibody for use according to any preceding claim, wherein tumor cells in the subject are classified according to (i) membrane FRα staining intensity on a scale from 0 (no detectable FRα) to 3 (high FRα) and (ii) percentage of tumor cells positive for FRα (from 0 to 100%); and wherein an overall tumor FRα expression score for the subject is determined as the product of (i) and (ii).

6. An IgE antibody for use according to claim 5, wherein the overall tumor FRα expression score for the subject is less than 100, preferably less than 50, more preferably less than 30.

7. An IgE antibody for use according to any preceding claim, wherein tumor FRα expression in the subject is lower than in at least 50% of cancer subjects; preferably wherein membrane FRα expression in tumor cells of the subject is lower than in at least 60%, at least 70% or at least 80% of subjects suffering from the same form of cancer; more preferably wherein tumor FRα expression in the subject is lower than in at least 50%, at least 70% or at least 90% of FRα-expressing tumors (preferably FRα-expressing ovarian tumors).

8. An IgE antibody for use according to any preceding claim, wherein the tumor expresses FRα; preferably wherein tumor cells in the subject show detectable membrane FRα expression by immunohistochemistry.

9. An IgE antibody for use according to any preceding claim, wherein at least 1% of tumor cells in the subject show detectable membrane FRα expression using immunohistochemical detection of FRα; preferably wherein at least 5%, 10%, 15% or 20% of tumor cells in the subject show detectable membrane FRα expression.

10. An IgE antibody for use according to any preceding claim, wherein the antibody is a MOv18 IgE antibody.

11. An IgE antibody for use according to any preceding claim, for use in treating and/or delaying progression of cancer in the subject.

12. An IgE antibody for use according to any preceding claim, wherein the tumor or cancer is an ovarian tumor or ovarian cancer.

13. An IgE antibody for use according to any preceding claim, wherein the antibody lacks a cytotoxic moiety, preferably wherein the antibody is not an antibody-drug conjugate (ADC).

14. An IgE antibody for use according to any preceding claim, wherein a maximum weekly dose of the IgE antibody administered to the subject is 50 mg, 25 mg, 10 mg, 3 mg or 1 mg.

15. An IgE antibody for use according to claim 14, wherein the weekly dose of the IgE antibody is 10 μg to 50 mg, 70 μg to 30 mg, 70 μg to 3 mg, 500 μg to 1 mg or about 700 μg.

16. An IgE antibody for use according to any preceding claim, wherein the IgE antibody is administered to the subject once a week or once every two weeks.

17. An IgE antibody for use according to claim 16, wherein the IgE antibody is administered to the subject for up to 12 weeks.

18. An IgE antibody according to claim 17, wherein the IgE antibody is administered to the subject (i) once a week for 6 weeks; followed by (ii) once every two weeks for 6 weeks.

19. An IgE antibody for use according to any preceding claim, wherein the IgE antibody is administered to the subject in a dose per administration of less than 1 mg/kg, less than 0.1 mg/kg or less than 0.03 mg/kg.

20. An IgE antibody for use according to any preceding claim, wherein the IgE antibody is administered to the subject in a dose of less than 1 mg/kg/week, less than 0.1 mg/kg/week, or less than 0.03 mg/kg/week.

21. A method for treating and/or delaying progression of cancer in a subject having a low FRα-expressing tumor, the method comprising a step of administering an anti-folate receptor alpha (FRα) immunoglobulin E (IgE) antibody as defined in any preceding claim to the subject in a therapeutically-effective amount.

22. A pharmaceutical composition for use in treating a low FRα-expressing tumor in a subject, comprising an anti-folate receptor alpha (FRα) immunoglobulin E (IgE) antibody as defined in any of claims 1 to 20 and one or more pharmaceutically acceptable excipients, carriers or diluents.

23. A pharmaceutical composition for use according to claim 22, wherein the composition comprises less than 50 mg of the IgE antibody.

24. A pharmaceutical composition for use according to claim 23, comprising less than 30 mg, less than 25 mg, less than 10 mg, less than 5 mg, less than 3 mg or less than 1 mg of the IgE antibody.

25. A pharmaceutical composition for use according to claim 23 or claim 24, comprising 10 μg to 50 mg, 70 μg to 30 mg, 70 μg to 3 mg, 500 μg to 1 mg or about 700 μg of the IgE antibody.

26. A pharmaceutical composition for use according to any of claims 22 to 25, wherein the composition is in the form of a liquid.

27. A pharmaceutical composition for use according to any of claims 22 to 26, comprising an aqueous solution having a concentration of 0.1 mg/ml to 10 mg/ml, 0.5 mg/ml to 2 mg/ml or about 1 mg/ml of the IgE antibody.

28. A pharmaceutical composition for use according to any of claims 22 to 27, wherein the pharmaceutically acceptable excipient is selected from sodium citrate, L-arginine, sucrose, polysorbate 20 and/or sodium chloride.

29. A pharmaceutical composition for use according to any of claims 22 to 28, wherein the composition is suitable for intravenous injection.

30. A composition according to claim 29, wherein the composition is suitable for intravenous injection up to a maximum total dose of 50 mg/week, 25 mg/week, 10 mg/week, 3 mg/week or 1 mg/week.


SEQUENCE LISTING (16 sequences)

SEQ ID NO: 1 - protein, 214 aa, Artificial Sequence
  MOv18 IgE Light (L) Chain
DIQMTQTTSSLSASLGDRVTISCRASQDINNFLNWYQQKPDGTVKLLIYYTSRLHSGVPSRFSGSGSGTDYSLTIINLEQEDIAIYFCQQSSTIPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 549 aa, Artificial Sequence
  MOv18 IgE Heavy (H) Chain
QVQLQQSGAELARPGASVKLSCKASDYIFTNYDITWVKQRPGQGLEWIGEIDPRSGKSYYNEKFKGKSTLTADKSSSTAYMELRSLTSEDSAVYFCATMYYYGSSPPMDYWGQGTSVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 3 - protein, 107 aa, Artificial Sequence
  MOv18 IgE Light Chain Variable Domain
DIQMTQTTSSLSASLGDRVTISCRASQDINNFLNWYQQKPDGTVKLLIYYTSRLHSGVPSRFSGSGSGTDYSLTIINLEQEDIAIYFCQQSSTIPRTFGGGTKLEIK

SEQ ID NO: 4 - protein, 121 aa, Artificial Sequence
  MOv18 IgE Heavy Chain Variable Domain
QVQLQQSGAELARPGASVKLSCKASDYIFTNYDITWVKQRPGQGLEWIGEIDPRSGKSYYNEKFKGKSTLTADKSSSTAYMELRSLTSEDSAVYFCATMYYYGSSPPMDYWGQGTSVTVSS

SEQ ID NO: 5 - protein, 257 aa, Homo sapiens
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 6 - DNA, 771 bp, Homo sapiens
atggctcagcggatgacaacacagctgctgctccttctagtgtgggtggctgtagtaggggaggctcagacaaggattgcatgggccaggactgagcttctcaatgtctgcatgaacgccaagcaccacaaggaaaagccaggccccgaggacaagttgcatgagcagtgtcgaccctggaggaagaatgcctgctgttctaccaacaccagccaggaagcccataaggatgtttcctacctatatagattcaactggaaccactgtggagagatggcacctgcctgcaaacggcatttcatccaggacacctgcctctacgagtgctcccccaacttggggccctggatccagcaggtggatcagagctggcgcaaagagcgggtactgaacgtgcccctgtgcaaagaggactgtgagcaatggtgggaagattgtcgcacctcctacacctgcaagagcaactggcacaagggctggaactggacttcagggtttaacaagtgcgcagtgggagctgcctgccaacctttccatttctacttccccacacccactgttctgtgcaatgaaatctggactcactcctacaaggtcagcaactacagccgagggagtggccgctgcatccagatgtggttcgacccagcccagggcaaccccaatgaggaggtggcgaggttctatgctgcagccatgagtggggctgggccctgggcagcctggcctttcctgcttagcctggccctaatgctgctgtggctgctcagc

SEQ ID NO: 7 - protein, 119 aa, Artificial Sequence
  Farletuzumab VH domain
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSS

SEQ ID NO: 8 - protein, 110 aa, Artificial Sequence
  Farletuzumab VL domain
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIK

SEQ ID NO: 9 - protein, 118 aa, Artificial Sequence
  huMOv19 VH domain
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSS

SEQ ID NO: 10 - protein, 112 aa, Artificial Sequence
  huMOV19 VL domain
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKR

SEQ ID NO: 11 - protein, 5 aa, Artificial Sequence
  huMOv19 VH-CDR1
GYFMN

SEQ ID NO: 12 - protein, 17 aa, Artificial Sequence
  huMOv19 VH-CDR2
RIHPYDGDTFYNQKFQG

SEQ ID NO: 13 - protein, 9 aa, Artificial Sequence
  huMOv19 VH-CDR3
YDGSRAMDY

SEQ ID NO: 14 - protein, 15 aa, Artificial Sequence
  huMOv19 VL-CDR1
KASQSVSFAGTSLMH

SEQ ID NO: 15 - protein, 7 aa, Artificial Sequence
  huMOv19 VL-CDR2
RASNLEA

SEQ ID NO: 16 - protein, 9 aa, Artificial Sequence
  huMOv19 VL-CDR3
QQSREYPYT